(19) United States
(12) Patent Application Publication
(10) Pub. No.: US 2026/0179222 A1
(43) Pub. Date: Jun. 25, 2026

(54) SYSTEMS AND METHODS FOR DIAGNOSING AND/OR TREATING PATIENTS

(71) Applicant: GI Scientific, LLC, Arlington, VA (US)

(72) Inventor: Scott Miller, Arlington, VA (US)

(21) Appl. No.: 19/460,191

(22) Filed: Jan. 26, 2026

Related U.S. Application Data
(63) Continuation of application No. 17/995,122.
(60) Provisional application No. 63/003,656, filed on Apr. 1, 2020.
(60) Provisional application No. 63/137,698, filed on Jan. 14, 2021.

(51) Int. Cl. G06T 7/00; A61B 1/00; G06V 10/25

(52) U.S. Cl. CPC G06T 7/0012; A61B 1/000096; A61B 1/00096; A61B 1/00101; A61B 1/00137; G06V 10/25; G06T 2207/10068; G06T 2207/20081; G06V 2201/03

(57) ABSTRACT

Devices, systems, and methods are provided for recognizing, diagnosing, mapping, sensing, monitoring and/or treating selected areas within a patient's body. The systems, devices and methods may be used to map, detect and/or quantify images and/or physiological parameters collected from the patient. One such system comprises an optical imaging device, such as an endoscope, and a processor coupled to the imaging device. The processor includes a software application configured to recognize the images captured by the optical imaging device and determine if the tissue contains a medical condition and may include an artificial neural network configured to develop at least one set of computer-executable rules useable to recognize the medical condition in the captured tissue images. The systems, devices and methods provided herein allow for a more objective and comprehensive inspection of the targeted areas within a patient so as to improve the diagnosis and ultimate treatment of patients.

### CROSS-REFERENCE TO RELATED APPLICATIONS

This application is a continuation of U.S. Nonprovisional Application No. Ser. No. 17/995,122, filed Sep. 30, 2022; which is a 371 Application of PCT/US 2021/025272, filed Mar. 31, 2021; which claims the benefit of U.S. Provisional Application Nos. 63/003,656, filed Apr. 1, 2020 and 63/137,698, filed Jan. 14, 2021, the entire disclosures of which are incorporated herein by reference for all purposes as if copied and pasted herein.

### FIELD OF THE INVENTION

The present disclosure relates to systems, methods and devices for recognizing and/or diagnosing disorders, diseases and other medical conditions and for mapping, treating and/or monitoring selected areas within a patient's body, such as the GI tract.

### BACKGROUND OF THE INVENTION

Recent advances in optical imaging technology have allowed many medical procedures to be performed today in a minimally invasive manner. The evolution of the more sophisticated, flexible scope with advanced visual capabilities has allowed access to regions deep within the human body that could only be achieved before with invasive surgical intervention. This modern day convenience has resulted in an increase in the demand for, as well as the number of, endoscopic, laparoscopic, arthroscopic, ophthalmoscopic, or other remote imaging visualization procedures performed every year in the U. S and globally. While these procedures are relatively safe, they are not without risks.

Endoscopy, for instance, is a procedure in which a lighted visualization device called an endoscope is inserted into the patient's body to look inside a body cavity, lumen, organ or in combination, for the purpose of examination, diagnosis or treatment. The endoscope may be inserted through a small incision or through a natural opening of the patient. In a bronchoscopy, the endoscope is inserted through the mouth, while in a sigmoidoscopy, the endoscope is inserted through the rectum. Unlike most other medical imaging devices, endoscopes are inserted directly into the organ, body cavity or lumen.

Today, most endoscopes are reused. This means that, after an endoscopy, the endoscope goes through a cleaning, disinfecting or sterilizing, and reprocessing procedure to be introduced back into the field for use in another endoscopy on another patient. In some cases, the endoscope is reused several times a day on several different patients.

While the cleaning, disinfecting and reprocessing procedure is a rigorous one, there is no guarantee that the endoscopes will be absolutely free and clear of any form of contamination. Modern day endoscopes have sophisticated and complex optical visualization components inside very small and flexible tubular bodies, features that enable these scopes to be as effective as they are in diagnosing or treating patients. However, the tradeoff for these amenities is that they are difficult to clean because of their small size, and numerous components. These scopes are introduced deep into areas of the body which expose the surfaces of these scopes to elements that could become trapped within the scope or adhere to the surface, such as body fluids, blood, and even tissue, increasing the risk of infection with each repeated use.

Endoscopes used in the gastrointestinal tract, such as forward viewing scopes, endoscopic ultrasound scopes (EUS) and duodenoscopes with side-viewing capability, have an added complexity in that they are in a bacteria rich environment. Typical gastroscopes, colonoscopes, duodenoscopes and EUS scopes have a camera lens, light and working channels with distal openings exposed to the patient environment. These elements of the scope all create cleaning issues, including the risk that bacteria finds its way into the working channel and other hard to clean locations on the scope. This provides an opportunity for bacteria to colonize and become drug resistant, creating the risk of significant illness and even death for a patient. This infection risk is also present in the cable mechanisms that are used to articulate instruments passing through the working channel and in other aspects of current scope designs. Moreover, in addition to the health risks posed by bacterial contamination and patient-to-patient cross-contamination, the accumulation of fluid, debris, bacteria, particulates, and other unwanted matter in these hard-to-clean areas of the scope also impact performance, shortening the useful life of these reusable scopes.

To reduce infection risks and protect the working end of endoscopes, disposable optical coupler devices have been designed for covering and at least partially sealing a portion of existing endoscopes. These coupler devices typically attach to the working end of the endoscope and have a visualization section composed of an optical material, such as glass, polycarbonate, acrylic, a clear gel or silicone, or other material with sufficient optical clarity to transmit an image, and which generally align with the camera lens and light source of the scope to allow for light to pass through the visualization section to provide a view of the target site by the endoscope.

While the recent advances in endoscopes and endoscope accessory or companion devices, such as optical couplers, have significantly improved the diagnosis and treatment of patient disorders, further advances in the capture and analysis of patient data during these procedures is warranted. For example, endoscopists may complete an examination without realizing that they have not taken complete images of the entire area sought to be examined. In such case, certain disorders within the patient may not be imaged and diagnosed, or the endoscopist may misdiagnose the patient due to incomplete information.

In addition, endoscopy is still largely a procedure that involves the subjective visual inspection of selected areas within a patient. When making an endoscopic diagnosis, a medical practitioner attempts to detect all predetermined detection targets that are to be carefully observed, such as a lesion or tumor in an organ. The accuracy of detecting these target sites is influenced by the experience, skill and sometimes by the degree of fatigue of the medical practitioner. Until recently, it usually took significant time and effort for endoscopists to learn about the many gastrointestinal diseases and train in the endoscopic detection and diagnosis of disorders, such as polyps, abnormal or diseased tissue, inflammation, malignant or benign tumors and the like. Even when the endoscopists are experts, however, they might sometimes miss the detection and diagnosis of disorders due to, for example, a similar color of the tissue to the surrounding area, the small size of the disorder, difficult locations of disorders, such as behind the folds of tissue within the GI tract, and other factors.

Accordingly, it is desirable to provide improved systems and methods for recognizing, monitoring, diagnosing and treating disorders, diseases and other medical conditions within a patient's body. It is particularly desirable to provide systems and methods for performing a more objective and comprehensive inspection of the targeted areas within a patient so as to improve the diagnosis and ultimate treatment of patients.

### SUMMARY OF THE INVENTION

The present disclosure is drawn to devices, systems, and methods for recognizing, diagnosing, monitoring and/or treating selected areas within a patient's body. In particular, in at least some aspects, the devices, systems and methods of the present disclosure may be used to analyze, recognize, diagnose, monitor, treat and/or predict medical conditions of tissue or other matter by detecting and objectively quantifying images and physiological parameters in a patient's body, such as the size, depth and overall topography of tissue, tissue biomarkers, tissue bioimpedance, temperature, PH, histological parameters, lesions or ulcers, bleeding, stenosis, pathogens, abnormal or diseased tissue, cancerous or precancerous tissue and the like. The medical conditions may include a variety of different tissue disorders, including, but not limited to, tumors, polyps, lesions, ulcers, inflammation, bleeding, stenosis, pathogens, abnormal or diseased tissue, cancerous or precancerous tissue and the like.

In one aspect, a system comprises an imaging device, such as an endoscope, a capsule endoscope or other suitable imaging device, having an optical element for capturing images of a tissue in the patient, and a processor coupled to the imaging device. The processor includes one or more software applications with one or more sets of instructions to cause the processor to recognize the images captured by the imaging device and to determine if the tissue contains a medical disorder, disease or other condition.

In certain embodiments, the software application(s) are configured to compare the tissue images with data related to one or more medical disorders, images of certain medical disorders or other data related to such disorders, such as tissue color, texture, topography and the like. In an exemplary embodiment, the software application(s) or processor may include an artificial neural network (i.e., an artificial intelligence or machine learning application) that allows the processor to develop computer-exercisable rules based on the tissue images captured from the patient and the data related to certain medical disorders to thereby further refine the process of recognizing and/or diagnosing the medical disorder.

The imaging device may be any imaging device capable of taking images of tissue within, or on, a patient, such as optical, infrared, thermal, ultrasound, X-ray, magnetic resonance (e.g., MRI), computed tomography (CT) photoacoustic, nuclear imaging (e.g., PET) or other types of images. The imaging device may be configured to transmit images to a receiving device, either through a wired or a wireless connection. The imaging device may be, for example, a component of an endoscope system, a component of a tool deployed in a working port of an endoscope, a wireless endoscopic capsule, or one or more implantable monitors or other devices. In the case of an implantable monitor, such an implantable monitor may be permanently or temporarily implanted.

The system may further include a memory in the processor or another device coupled to the processor. In one such embodiment, the memory further contains images of representative tissue, and the processor is configured to compare the current images captured by the endoscope with the representative tissue. The memory may, for example, contain images of tissue from previous procedures on the same patient. In this embodiment, the processor is configured to compare the images taken during the current procedure with images from previous procedures. In some cases, these previous images include a topographic representation of an area of the patient, such as the GI tract or other selected area. The processor is further configured to determine, for example, if the physician has examined the entire area selected for examination (e.g., by comparing the current images with previous images that represent the entire area). The processor may make this determination in real-time to alert the physician that, for example, the examination has not been completed. In other embodiments, the processor may be configured to save the images so that the physician can confirm that the examination has been complete.

In other embodiments, the previous images may include selected tissue or areas from the patient, such as a medical disorder. The medical disorder may, for example, include a tumor, polyp, ulcer, inflammation, abnormal or diseased tissue or other disorder. In this embodiment, the processor comprises one or more software applications with sets of instructions that allow the processor to compare the current images of the disorder with previous images to, for example, determine if the disorder has changed between the procedures. For example, the software applications may have a set of instructions that compare previous and current images of cancerous tissue and then determine if the cancerous tissue has grown or changed in any material aspect. In another example, the processor may determine if a previously-removed polyp or tumor has returned or was completely removed in a previous procedure.

In other embodiments, the memory contains images of representative tissue from patients other than the current patient. For example, the representative tissue may comprise a series of images of certain types of disorders, such as a tumor, polyp, ulcer, inflammation or a diseased tissue. In this embodiment, the system further includes one or more software applications coupled to the processor and configured to characterize the disorder in the patient based on the images captured by the endoscope and the images of the representative tissue. The software applications may include an artificial neural network (e.g., an artificial intelligence or machine-learning program) that includes a set of instructions that allows the software applications to “learn” from previous images and apply this learning to the images captured from the patient. The software application can be used to, for example, supplement the physician's diagnosis of the disorder based on the series of images of other similar disorders and/or to reduce the variation in diagnostic accuracy among medical practitioners.

In certain embodiments, the software application may be configured to analyze images from the entire area of the procedure and compare these images with data or other images in the memory. The software application may be further configured to detect a potential disorder in the selected area of examination based on the images and data within memory. Detection of a potential disease or disorder by the software application during the endoscopic diagnosis makes it possible to prevent a detection target from being overlooked by a medical practitioner, thereby increasing the confidence of an endoscopic diagnosis.

In certain embodiments, the memory includes a variety of different patient characteristics that create a patient profile, such as age, ethnicity, nationality, race, height, weight, baseline vitals, such as blood pressure, heart rate and the like, hematology results, blood chemistry or urinalysis results, physical examinations, medication usage, blood type, BMI index, prior medical history (e.g., diabetes, prior cancerous events, irritable bowel syndrome or other GI tract issues, frequency of colonoscopies, frequency and growth rate of polyps, etc.) and other relevant variables. The memory may be linked to a central repository in a computer network or similar type network that provides similar profiles from a multitude of different patients in different locations around the country. In this manner, an individual health care practitioner or hospital staff can access hundreds or thousands of different patient profiles from various locations around the country or the world.

In this embodiment, the processor may include an artificial neural network capable of classifying the patient based on a comparison of his/her individual profile and the other profiles in the network. This classification may include a relevant risk profile for the patient to develop certain disorders or diseases. Alternatively, it may allow the software application(s) to recognize the medical disorder based on the images and/or data collected during the procedure.

The system may be configured to capture data relevant to the actual size and depth of tissue, lesions, ulcers, polyps, tumors and/or other abnormalities within the patient. For example, the size of a lesion or ulcer may range from a scale of 100 micrometers to a few centimeters. The software applications may include sets of instructions to cause the processor to collect this depth information and to classify the depth as being superficial, submucosal, and/or muscularis. The processor also be configured to capture data regarding the prevalence of impact of lesions or ulcers within a specific region of the patient.

Data gathered from any of the sources above may be used to train an algorithm, such as an AI algorithm, to predict exacerbations or flare-ups. Information input regarding medication may be used to, for example, predict or otherwise consider a patient's response to medication and enable a health care provider, patient, caregiver or other party to tailor medication treatments. Data from different sources described above may be combined in various permutations in order to enable predictive diagnostics and/or treatment recommendations.

In another aspect of the invention, a system for examining a patient comprises an endoscope having an optical element for capturing images of a selected area in the patient and a coupler device for use with the endoscope. The coupler device comprises a main body having a visualization section configured to allow viewing of the surgical site, and an attachment section having a proximal end configured for attachment to a distal end portion of the endoscope. The system further includes a processor coupled to the endoscope and having a memory for retaining the images captured by the endoscope. The processor further includes one or more software applications having a set of instructions for providing data related to the selected area based on the retained images.

In certain embodiments, the system may further include one or more sensors on, or within, an outer surface of the main body of the coupler device. The sensors are configured to detect a physiological parameter of tissue around the outer surface of the main body of the coupler device. The physiological parameter may include, for example, a temperature of the tissue. a dimension of the tissue, a depth of the tissue, tissue topography, tissue biomarkers, tissue bioimpedance, temperature, PH, histological parameters or another parameter that may be used for diagnosing a medical condition.

The system further includes a connector configured to couple the sensor to a processor. The processor may also receive images from the camera on the endoscope. In certain embodiments, the processor is configured to create a topographic representation of the tissue based on the images and/or the physiological parameter(s). In this embodiment, the system may further comprise a memory containing data regarding the physiological parameter from either the current patient or a plurality of other patients. The system includes a software application coupled to the processor and configured to diagnose the patient based on the physiological parameter detected by the sensor and the images captured by the endoscope. The software application may include an artificial neural network (e.g., an artificial intelligence or machine-learning program) that allows the software application to “learn” from previous physiological parameters of the patient, or from physiological parameters of other patients and then apply this learning to the data captured from the patient. The system may include, for example, a trained machine learning algorithm configured to develop from the images of representative tissue at least one set of computer-executable rules useable to recognize a medical condition in the tissue images captured by the endoscope. For example, the software application may be configured to diagnose one or more disease parameters based on the physiological parameter and/or the images.

In certain embodiments, the system may further include a companion or coupler device removably attached to a distal end portion of the endoscope. The coupler device preferably includes a visualization section for allowing viewing of the tissue site through the coupler device, and an attachment section for removably mounting the coupler device to the endoscope. The coupler device may further include one or more sensors on, or within, an outer surface of the main body of the coupler device. The sensors are configured to detect a physiological parameter of tissue around the outer surface of the main body of the coupler device. The physiological parameter may include, for example, a temperature of the tissue. a dimension of the tissue, a depth of the tissue, tissue topography, tissue biomarkers, tissue bioimpedance, temperature, PH, histological parameters or another parameter that may be used for diagnosing a medical condition.

In this embodiment, the system may further comprise a memory containing data regarding the physiological parameter from either the current patient or a plurality of other patients. The system includes a software application coupled to the processor and configured to diagnose the patient based on the physiological parameter detected by the sensor and the images captured by the endoscope. The software application may include an artificial neural network (e.g., an artificial intelligence or machine-learning program) that allows the software application to “learn” from previous physiological parameters of the patient, from physiological parameters and/or data of other patients and/or objective criteria related to the medical condition. The machine-learning program is configured to develop a set of computer-exercisable rules to apply this learning to the data captured from the patient. For example, the software application may be configured to diagnose one or more disease parameters based on the physiological parameters, the images or other data collected from the patient.

The coupler device also protects the scope and its components, particularly the scope elevator, to reduce the risk of debris, fluid and other matter ending up in the elevator and behind the elevator and the working or biopsy channel, potentially causing infection risk. In certain embodiments, the coupler device includes an open area, cavity or channel that allows the instrument to pass through the coupler device to the surgical site. The instrument(s) may be articulated by a variety of suitable means, such as cables, elevators, piezo electric materials, micro motors, organic semiconductors, electrically activated polymers or other sources of energy or power, that are either disposed within the coupler device, on or within the endoscope, or external to both and suitably coupled to the instrument(s).

In other embodiments, the coupler device includes a flexible working channel extension that extends the working or biopsy channel of the scope and can be angularly adjustable. The flexible working channel extension may be adjustable by an elevator or cable passing through the endoscope. Alternatively, the coupler device may include its own actuator, such as an elevator, cable, or similar actuation means, for adjusting the working channel extension and thereby articulating instruments passing through the endoscope. The actuator may be powered by any suitable source of energy, such as a motor or the like. The source of energy may be coupled to the actuator either directly through the scope, or indirectly through magnetic, electric, or some other source of energy. The source of energy may be disposed within the coupler device, or it may be external to the coupler device (i.e., either disposed on the proximal end of the scope or external to the patient).

The coupler device may be provided as a single-use disposable accessory to an endoscope that provides the user with the ability to change the angle of exit of a device being advanced out of the working channel of an endoscope, without exposing the distal end of the scope to bacteria, debris, fluid and particulate matter. In some embodiments, the device attaches to the end of the endoscope and covers the working channel of the endoscope with a working channel extension in the coupler device, allowing an instrument to be passed down the working channel of the endoscope and into the working channel extension of the coupler device. The working channel extension can provide a seal against the scope working channel, so instruments can be passed back and forth through the scope working channel and out the working channel extension of the coupler device without fluid and bacteria entering areas outside of the scope working channel. This seal is accomplished, in some embodiments, through an extension of the device working channel into the scope working channel, through a gasket on the end of the working channel extension, by way of a temporary glue, through pressure and the seal of the overall device against the distal end of the scope, through the selection of elastic and elastomeric materials, and other suitable and alternative means.

In some embodiments, the device allows the user to articulate the working channel of the device in the direction preferred by the user of the endoscope, so that a wire, catheter or other instrument being advanced down the working channel of the endoscope can direct the wire or catheter or other instrument in a preferred direction different than the angle at which the instrument would exit the endoscope if the coupler device was not in place or if an elevator in the scope is not used. This redirection of an instrument has the benefit of assisting with the navigation of the device, while not allowing fluid, debris, particulate matter, bacteria and other unwanted elements to enter hard to clean areas of the endoscope, especially at the distal end of the endoscope.

In some embodiments, the device may be integrated into a scope and configured to be detachable and reusable for separate cleaning, including manual cleaning, in an autoclave, an ETO sterilizer, gamma sterilizer, and other sterilization methods.

In some embodiments, the coupler device may cover the entire distal end of the endoscope, or may just cover hard to clean areas. In some embodiments, the coupler device may cover the distal end of the endoscope, or a portion thereof, or it may include a sheath attached to the coupler device which covers the entirety of the scope that is exposed to fluid, debris, particulate matter, bacteria and other unwanted elements.

In another aspect of the invention, a system for diagnosing a disorder in a patient comprises an endoscope having an optical element for capturing images of a surgical site in the patient and a tissue or fluid sample extractor coupled to the endoscope and configured to withdraw a tissue or fluid sample from the surgical site. The system further includes an ionizer coupled to the sample extractor and configured to convert a portion of the tissue or fluid sample from the patient into ions. A mass analyzer is coupled to the ionizer and configured to sort the ions, preferably based on a mass-to-charge ratio, and a detector is coupled to the mass analyzer and configured to measure a quantity of each of the ions after they have been sorted.

In certain embodiments, the system further comprises a processor coupled to the detector having one or more software applications with a set of instructions to characterize a medical condition of the patient based on the quantity of each of the ions in the tissue sample. The medical condition may include a variety of disorders such as tumors, polyps, ulcers, diseased tissue, pathogens, cancerous or precancerous tissue or the like. In one embodiment, the medical condition comprises a tumor and the processor is configured to diagnose the tumor based on the quantity of each of the ions retrieved from the tissue sample. For example, the processor may be configured to determine the type of proteins or peptides existing in a tissue sample based on the type and quantity of ions. Certain proteins or peptides may provide information to the processor that the tissue sample is, for example, cancerous or pre-cancerous.

In certain embodiments, the system further includes a coupler device for use with the endoscope, the coupler device comprising a main body having a visualization section configured to allow viewing of the surgical site, and an attachment section having a proximal end configured for attachment to a distal end portion of the endoscope. In these embodiments, the extractor may be attached to the coupler device or the endoscope. The system may further comprise a connector on the endoscope or the coupler device for coupling the extractor with the ionizer and an aspirator coupled to the connector for withdrawing the tissue sample through the connector to the ionizer.

In certain embodiments, the endoscope comprises a tissue withdrawal lumen having a proximal end coupled to the mass analyzer and a distal end at or near the distal end of the scope. The tissue withdrawal lumen is further coupled to an aspiration device configured to aspirate tissue and/or fluid from a target site on, or within, a patient. The tissue withdrawal lumen may further include a fluid delivery system and/or a gas delivery system for delivering fluid and/or gas to the target site to collect the sample tissue or fluid from the patient. In a preferred embodiment, the fluid delivery system is configured to deliver one or more water droplets to the target site to collect molecules from the sample tissue.

The ionizer may include a heater for vaporizing the tissue or fluid sample and an electron source for ionizing the vaporized tissue. The heater and/or electron source may be located on the endoscope, the optical coupler or external to both. In the latter embodiment, the tissue sample is withdrawn through the endoscope from the patient before it is vaporized and ionized. In the former embodiment, the tissue sample is vaporized and/or ionized in-situ.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a schematic view of a system for monitoring, mapping, diagnosing, treating and/or evaluating tissue within a patient;

FIG. 2 is a partial cross-sectional view of the proximal portion of a representative endoscope coupled to a representative processor according to the present disclosure;

FIG. 3 is a perspective view of the distal end portion of a side-viewing endoscope according to the present disclosure;

FIGS. 4A and 4B are isometric views of an exemplary embodiment of the coupler device of the present disclosure in use with a duodenum scope.

FIGS. 5A and 5B show partial cutaway views of the coupler device and a duodenum scope of FIGS. 4A and 4B, respectively.

FIG. 6 shows another cutaway view of the coupler device and a duodenum scope of FIGS. 4A and 4B.

FIG. 7 shows still another cutaway view of the coupler device and a duodenum scope of FIGS. 4A and 4B.

FIG. 8 is a cutaway side view of the coupler device and a duodenum scope of FIGS. 4A and 4B in a first position.

FIG. 9 is a cutaway side view of the coupler device and a duodenum scope of FIGS. 4A and 4B in a second position.

FIG. 10 is a cutaway side view of the coupler device and a duodenum scope of FIGS. 4A and 4B in a third position.

FIG. 11 is an enlarged side view of the working channel extension with membrane of the coupler device of FIGS. 4A and 4B.

FIG. 12 is a side view of an alternative embodiment of an optical coupler according to the invention.

FIG. 13 is a cross-sectional view of the optical coupler of FIG. 12 taken along line 2-2 of FIG. 12.

FIG. 14 is a cross-sectional view of the optical coupler of FIGS. 12 and 13 taken along line 3-3 of FIG. 13, the optical coupler being attached to an endoscope.

FIG. 15 is a cross-sectional view of a second embodiment of an optical coupler according to the invention, the optical coupler being attached to an endoscope.

FIG. 16 is a cross-sectional view of the second embodiment of the optical coupler according to the invention engaging an inner wall of a body cavity.

FIG. 17 is a cross-sectional view of the second embodiment of the optical coupler according to the invention engaging an inner wall of a body cavity wherein a medical instrument has been advanced through an instrument lumen of the endoscope, an instrument channel of the optical coupler, a solid body of the optical coupler, and against the inner wall of the body cavity.

FIG. 18 is a cross-sectional view of a third embodiment of an optical coupler according to the present disclosure incorporating a tissue collection lumen.

FIG. 19 is a cross-sectional view of a distal end portion of the tissue collection lumen of the optical coupler of FIG. 18.

FIG. 20 is schematic view of a particle detection system according to the present disclosure.

### DETAILED DESCRIPTION OF THE INVENTION

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

The present disclosure is drawn to devices, systems, and methods for recognizing, diagnosing, mapping, sensing, monitoring and/or treating selected areas within a patient's body. In particular, in at least some aspects, the devices, systems and methods of the present disclosure may be used to diagnose, monitor, treat and/or predict tissue conditions by mapping, detecting and/or quantifying images and physiological parameters in a patient's body, such as size, depth and overall topography of tissue, biomarkers, bioimpedance, temperature, PH, histological parameters, lesions or ulcers, bleeding, stenosis, pathogens, diseased tissue, cancerous or precancerous tissue and the like. The devices, systems, and methods of the present disclosure may be used to monitor, recognize and/or diagnose a variety of conditions including, but not limited to, gastrointestinal conditions such as nausea, abdominal pain, vomiting, pancreatic, gallbladder or biliary tract diseases, gastrointestinal bleeding, irritable bowel syndrome (IBS), gallstones or kidney stones, gastritis, gastroesophageal reflux disease (GERD), inflammatory bowel disease (IBD), Barrett's esophagus, Crohn's disease, polyps, cancerous or precancerous tissue or tumors, peptic ulcers, dysphagia, cholecystitis, diverticular disease, colitis, celiac disease, anemia, and the like.

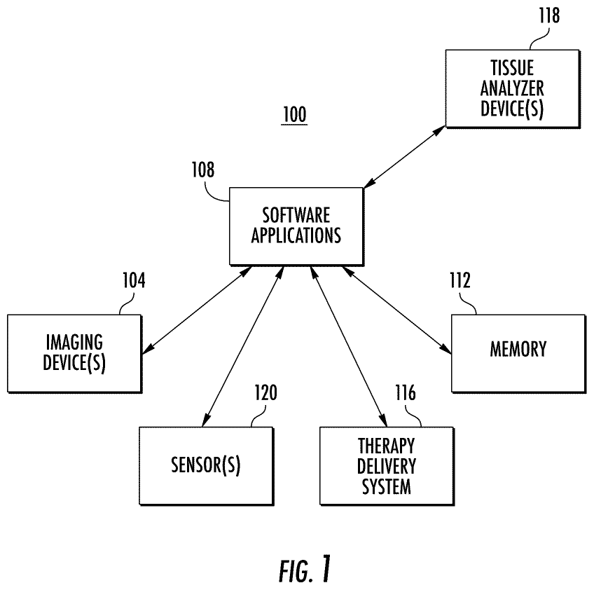

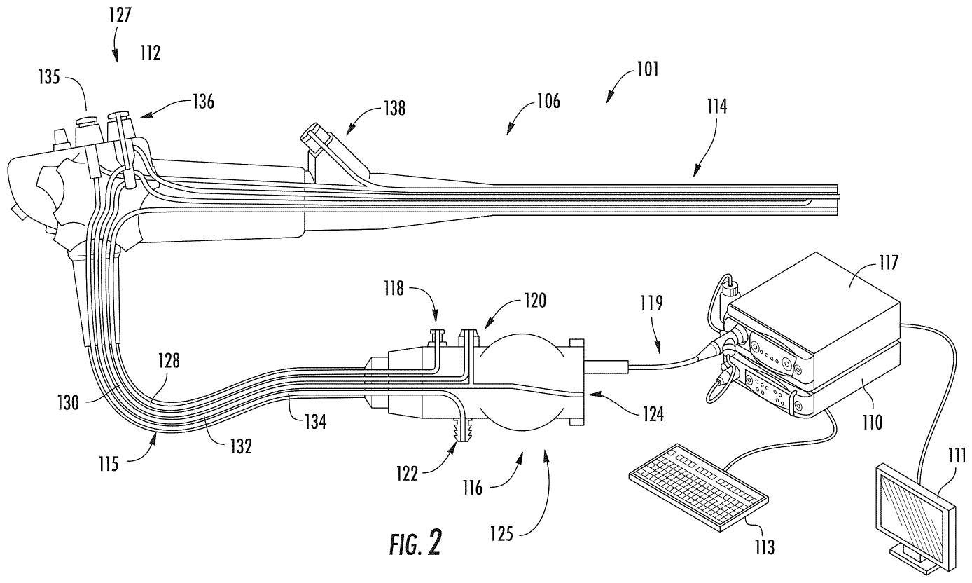

FIG. 1 depicts an exemplary diagnostic, mapping, treating and/or monitoring system 100. Monitoring system 100 may include, among other things, one or more imaging devices 104, one or more software applications 108, a memory 112, one or more therapy delivery systems 116, one or more tissue analyzing devices 118 and one or more sensors 120 that may be incorporated into the imaging devices 104, therapy delivery systems 116 or both. Software applications 108 include one or more algorithms that include sets of instructions to allow a processor (see FIG. 2) to build a model based on the data obtained from the patient by sensors 120, imaging devices 104, tissue analyzing devices 118 and/or certain data stored within memory 112.

In certain embodiments, memory 112 may contain images and/or data captured during a procedure on a patient. Memory 112 may also contain images and/or data of representative tissue, such as images and/or data of tissue from previous procedures on the same patient. In some cases, these previous images include a topographic representation of an area of the patient, such as the GI tract or other selected area. In other embodiments, the previous images may include selected tissue or areas from the patient, such as a medical disorder. In other embodiments, memory 112 contains images and/or data of representative tissue from patients other than the current patient. For example, the representative tissue may comprise a series of images of certain types of disorders, such as a tumor, polyp, ulcer, inflammation or abnormal or diseased tissue. These images may, for example, include hundreds or even thousands of different images of certain types of disorders (e.g., a particular type or grade of cancerous tissue). These images are available for software applications 108 to compare against the images collected by imaging devices 104 to facilitate the recognition of a disorder in the patient, as discussed in more detail below.

Software application(s) 108 include sets of instructions to allow processor 102 to analyze signals from imaging device 104 and other inputs, such as sensors 120, medical records, medical personnel, and/or personal data; and extract information from the data obtained by imaging device 104 and the other inputs. Processor 102 or any other suitable component may apply an algorithm with a set of instructions to the signals or data from imaging device 104, sensors 120 and other inputs. Processor 102 may store information regarding algorithms, imaging data, physiological parameters of the patient or other data in memory 112. The data from inputs such as imaging device 104 may be stored by processor 102 in memory 112 locally on a specialized device or a general-use device such as a smart phone or computer. Memory 112 may be used for short-term storage of information. For example, memory 112 may be RAM memory. Memory 112 may additionally or alternatively be used for longer-term storage of information. For example, memory 112 may be flash memory or solid state memory. In the alternative, the data from imaging device 104 may be stored remotely in memory 112 by processor 102, for example in a cloud-based computing system.

In certain embodiments, software applications 108 may be aided by an artificial neural network (e.g., machine learning or artificial intelligence). Machine learning is the scientific study of algorithms and statistical models that computer systems use to perform a specific task without using explicit instructions, relying on patterns and inference instead. Machine learning algorithms build a mathematical model based on sample data, known as “training data”, in order to make predictions or decisions without being explicitly programmed to perform the task. The artificial neural network may use algorithms, heuristics, pattern matching, rules, deep learning and/or cognitive computing to approximate conclusions without direct human input. Because the AI network can identify meaningful relationships in raw data, it can be used to support diagnosing, treating and predicting outcomes in many medical situations.

The artificial neural network includes one or more trained machine learning algorithms that process the data received from imaging devices 104 and sensors 120 and compares this data with data within memory 112. The artificial neural network may, for example, compare data and/or images collected from other patients on certain disorders and compare this data and/or images with the images collected from the patient. The artificial neural network is capable of recognizing medical conditions, disorders and/or diseases based on this comparison. In another example, the artificial neural network may combine data within memory 112 with images taken from the target site(s) of the patient to create a two or three dimensional map of the topography of a certain area of the patient, such as the gastrointestinal tract. In yet another example, the algorithms may assist physicians with interpretation of the data received from sensors 120 and/or imaging device 104 to diagnose disorders within the patient.

In one embodiment, software application(s) 108 include sets of instructions for the processor 102 to compare the images captured by imaging device 104 with the representative tissue in memory 112. Memory 112 may, for example, contain images and/or data of tissue from previous procedures on the same patient. In this embodiment, software application(s) 108 include sets of instructions for processor 102 to compare the images taken during the current procedure with images from previous procedures. In some cases, these previous images include a topographic representation of an area of the patient, such as the GI tract or other selected area. Software application 108 may have further sets of instructions for processor 102 to determine, for example, if the physician has examined the entire area selected for examination (e.g., by comparing the current images with previous images that represent the entire area). The processor 102 may make this determination in real-time to alert the physician that, for example, the examination has not been completed. In other embodiments, software application(s) 108 may have sets of instructions for the processor 102 to save the images in memory 112 so that the physician can confirm that the examination has been complete.

In other embodiments, the previous images may include selected tissue or areas from the patient, such as a medical disorder. The medical disorder may, for example, include a tumor, polyp, ulcer, inflammation, diseased tissue or other disorder. In this embodiment, software application(s) 108 include sets of instructions for the processor 102 to compare the current images of the disorder with previous images in memory 112 to, for example, allow the medical practitioner to determine if the disorder has changed between the procedures. For example, processor 102 may determine if a cancerous tissue has grown or changed in any material aspect. In another example, processor 102 may determine if a previously-removed polyp or cancerous tissue has returned or was completely removed in a previous procedure.

In other embodiments, memory 112 contains images and/or data of representative tissue from patients other than the current patient. For example, the representative tissue may comprise a series of images of certain types of disorders, such as a tumor, polyp, ulcer, lesion, inflammation or a cancerous or otherwise diseased tissue. In this embodiment, software application(a) 108 include a set of instructions for processor 102 to recognize and diagnose the disorder in the patient based on the images captured by imaging device 104 and the images of the representative tissue. Processor 102 may include an artificial neural network (e.g., an artificial intelligence or machine-learning program) that allows software application(s) 108 to “learn” from previous images and apply this learning to the images captured from the patient. Software application(s) 108 can be used to, for example, supplement the physician's diagnosis of the disorder based on the series of images of other similar disorders and/or to reduce the variation in diagnostic accuracy among medical practitioners.

In certain embodiments, software application(s) 108 may include sets of instructions for processor 102 to analyze images from the entire area of the procedure and compare these images with data or other images in memory 112. Software application(s) 108 may include further sets of instructions for processor 102 to detect a potential disorder in the selected area of examination based on the images and data within memory 112. Detection of a potential disease or disorder by software application 108 during the endoscopic diagnosis makes it possible to prevent a detection target from being overlooked by a medical practitioner, thereby increasing the confidence of an endoscopic diagnosis.

In certain embodiments, memory 112 includes a variety of different patient characteristics that create a patient profile, such as age, ethnicity, nationality, race, height, weight, baseline vitals, such as blood pressure, heart rate and the like, hematology results, blood chemistry or urinalysis results, physical examinations, medication usage, blood type, BMI index, prior medical history (e.g., diabetes, prior cancerous events, irritable bowel syndrome or other GI tract issues, frequency of colonoscopies, frequency and growth rate of polyps, etc.) and other relevant variables. Memory 112 may be linked to a central repository in a computer network or similar type network that provides similar profiles from a multitude of different patients in different locations around the country. In this manner, an individual health care practitioner or hospital staff can access hundreds or thousands of different patient profiles from various locations around the country or the world.

In this embodiment, software application 108 may include an artificial neural network capable of classifying the patient based on a comparison of his/her individual profile and the other profiles in the network. This classification may include a relevant risk profile for the patient to develop certain disorders or diseases. Alternatively, it may allow the software application 108 to diagnose the patient based on the images and/or data collected during the procedure.

In another embodiment, software application 108 and memory 112 are configured to maintain records of a particular health care provider (e.g., endoscopist) and/or health center (e.g., hospital, ASC or the like) related to the procedures performed by that health care provider or health center. These records may, for example, include the number of colonoscopies performed by a health care provider, the results of such procedures (e.g., detection of a disorder, time spent for the procedure and the like). Software application 108 is configured to capture the data within memory 112 and compute certain attributes for each particular health care provider or health center. For example, software application 108 may determine a disorder detection rate of a particular health care provider and compare that rate versus other health care providers or health centers.

Certain institutions, such as health insurance companies, may be particularly interested in comparing such data across different health care providers or health centers. For example, software application 108 may be configured to measure the adenoma detection rate of a particular health care provider or health center and compare that rate to other health care providers or to an overall average that has been computed from the data in memory 112. This adenoma detection rate can, for example, be used to profile a health care provider or, for example, as a quality control for insurance purposes.

In certain embodiments, the processor and/or software applications 108 are configured to record the time throughout the procedure and to capture the exact time of certain events during the procedure, such as the start time (i.e., the time the endoscope is advanced into the patient's body), the time that the endoscope captures images of certain disorders or certain target areas within the patient, the withdrawal time and the like. Software application 108 is configured to measure, for example, the time spent for the entire procedure, the time spent from entry into the patient to image capture of a certain disorder and the like. This data can be collected into memory 112 for later use. For example, an insurance provider may desire to know the amount of time a surgeon spends in a procedure or the amount of time it takes from entry into the patient until the surgeon reaches a particular disorder, such as a lesion, tumor, polyp or the like.

Data gathered from any of the sources above may be used to train an algorithm, such as an AI algorithm, to predict exacerbations or flare-ups. Information input regarding medication may be used to, for example, predict or otherwise consider a patient's response to medication and enable a health care provider, patient, caregiver or other party to tailor medication treatments. Data from different sources described above may be combined in various permutations in order to enable predictive diagnostics and/or treatment recommendations.

The artificial neural network within processor 102 may be configured to perform a difference analysis between the images captured by imaging device 104 and a prediction image. The prediction image may be generated based on images of representative tissue within memory 112 or other tissue data that has been downloaded onto processor 102. The difference analysis may include, but is not limited to, comparing textures, colors, sizes, shapes, spectral variations, biomarkers, or other characteristics of the images captures by imaging device 104 and the prediction image.

In certain embodiments, diagnostic system 100 is part of a larger network that may include hundreds or thousands of other systems similar to system 100. In this embodiment, when system 100 recognizes a medical condition or disorder and provides a preliminary diagnosis of that condition or disorder, this information may be communicated back to a central processor or computer server (not shown) that is managed as part of a proprietary system. This information may be accumulated from multiple independent users of the system located in remote locations (i.e., different hospitals around the country). The accumulated data may be examined for quality control and then added to a larger database. This added data may be used to further calibrate and fine-tune the overall system for improved performance. The artificial neural network continually updates memory 112 and software application(s) 108 to improve the accurate of diagnosis of these disorders.

In addition, the artificial neural network in processor 102 may be configured to generate a confidence value for the diagnosis of a particular disorder or disease. The confidence level may, for example, illustrate a level of confidence that the disease is present in the tissue based on the images taken thereof. The confidence value(s) may also be used, for example, to illustrate overlapping disease states and/or margins of the disease type for heterogenous diseases and the level of confidence associated with the overlapping disease states.

In certain embodiments, the artificial neural network in processor 102 may include sets of instructions to grade certain diseases, such as cancer. The grade may, for example, provide a degree of development of the cancer from an early stage of development to a well-developed cancer (e.g., Grade 1, Grade 2, etc.). In this embodiment, software application(s) 108 include a set of instructions for processor 102 to compare the characteristics of an image captured by imaging device 104 with data from memory 112 to provide such grading.

In addition, system 100 may include a set of instructions for processor 102 to distinguish various disease types and sub-types from normal tissue (e.g., tissue presumed to have no relevant disease). In this embodiment, system 100 may differentiate normal tissue proximal to a cancerous lesion and normal tissue at a distal location from the cancerous lesion. The artificial neural network may be configured to analyze the proximal normal tissue, distal normal tissue and benign normal tissue. Normal tissue within a tumor may have a different signature than benign lesions and proximal normal tissue may have a different signature than distal normal tissue. For example, the signature of the proximal normal tissue may indicate emerging cancer, while the signature in the distal normal tissue may indicate a different disease state. In this embodiment, system 100 may use the proximity of the tissue to the cancerous tissue to, for example, measure a relevant strength of a disease, growth of a disease and patterns of a disease.

Sensor(s) 120 are preferably disposed on, or within, one or more of the imaging devices 104. In certain embodiments, sensors 120 are located on a distal end portion of an endoscope (discussed below). In other embodiments, sensors 120 are located on, or within, a coupling device (such as coupling device 10 or optical coupler 200 discussed below) attached to the distal end portion of the endoscope.

Sensor(s) 120 are configured to detect one or more physiological parameter(s) of tissue around the outer surface of the main body. The physiological parameter(s) may include a temperature of the tissue, a type of fluid in, or around, the tissue, pathogens in, or around, the tissue, a dimension of the tissue, a depth of the tissue, a tissue disorder, such as a lesion, tumor, ulcer, polyp or other abnormality, biological receptors in, or around, the tissue, tissue biomarkers, tissue bioimpedance, a PH of fluid in, or around the tissue or the like.

In certain embodiments, the sensor(s) 120 detect temperature of the tissue and transmit this temperature data to the processor. Software applications 108 include a set of instructions to compare the tissue temperature with data in memory 112 related to standard tissue temperature ranges. Processor is then able to determine if the tissue includes certain disorders based on the tissue temperature (e.g., thermography). For example, certain tumors are more vascularized than ordinary tissue and therefore have higher temperatures. The memory 112 includes temperature ranges that indicate “normal tissue” versus highly vascularized tissue. The processor can determine if the tissue is highly vascularized based on the collected temperature to indicate that the tissue may be cancerous.

In certain embodiments, sensor(s) 120 may include certain components configured to measure the topography of the tissue near the surface of the coupler device. For example, sensor(s) 120 may be capable of providing a 3-D representation of the target tissue. In certain embodiments, sensor(s) 120 are capable of measuring reflected light and capturing information about the reflected light, such as the return time and/or wavelengths to determine distances between the sensor(s) 120 and the target tissue. This information may be collected by software application 108 to create a digital 3-D representation of the target tissue.

In one embodiment, the coupler device 10, optical coupler 200 or the endoscope further includes a light imaging device that uses ultraviolet, visible and/or near infrared light to image objects. The light may be concentrated into a narrow beam to provides very high resolutions. The light may be transmitted with a laser, such as a YAG laser, holmium laser and the like. In one preferred embodiment, the laser comprises a disposable or single-use laser fiber mounted on or within the optical coupler device. Alternatively, the laser may be advanced through the working channel of the endoscope and the optical coupler device.

Sensor(s) 120 are capable of receiving and measuring the reflected light from the laser (e.g., LIDAR or LADAR) and transmitting this information to the processor. In this embodiment, one or more software applications 108 are configured to transform this data into a 3-D map of the patient's tissue. This 3-D map may can be used to assist with the diagnosis and/or treatment of disorders in the patient.

In another embodiment, monitoring system 100 includes an ultrasound transducer, probe or other device configured to produce sound waves and bounce the sound waves off tissue within the patient. The ultrasound transducer receives the echoes from the sound waves and transmits these echoes to the processor. The processor includes one or more software applications 108 with a set of instructions to determine tissue depth based on the echoes and/or produce a sonogram representing the surface of the tissue. The ultrasound probe may be delivered through a working channel in the endoscope and the optical coupler device. Alternatively, the transducer may be integrated into either the endoscope or the optical coupler device. In this latter embodiment, the transducer may be, for example, a disposable transducer within the optical coupler device that receives electric signals wirelessly, or through a connector extending through the endoscope.

Suitable sensors 120 for use with the present invention may include PCT and microarray based sensors, optical sensors (e.g., bioluminescence and fluorescence), piezoelectric, potentiometric, amperometric, conductometric, nanosensors or the like. Physical properties that can be sensed include temperature, pressure, vibration, sound level, light intensity, load or weight, flow rate of gases and liquids, amplitude of magnetic and electronic fields, and concentrations of many substances in gaseous, liquid, or solid form. Sensors 120 can measure anatomy and movement in three dimensions using miniaturized sensors, which can collect spatial data for the accurate reconstruction of the topography of tissue in the heart, blood vessels, gastrointestinal tract, stomach, and other organs. Pathogens can also be detected by another biosensor, which uses integrated optics, immunoassay techniques, and surface chemistry. Changes in a laser light transmitted by the sensor indicate the presence of specific bacteria, and this information can be available in hours

Sensors 120 can measure a wide variety of parameters regarding activity of the selected areas in the patient, such as the esophagus, stomach, duodenum, small intestine, and/or colon. Depending on the parameter measured, different types of sensors 120 may be used. For example, sensor 120 may be configured to measure pH via, for example, chemical pH sensors. Gastric myoelectrical activity may be measured via, for example, electrogastrography (“EGG”). Gastric motility and/or dysmotility may be measured, via, for example, accelerometers, gyroscopes, pressure sensors, impedance gastric motility (IGM) using bioimpedance, strain gauges, optical sensors, acoustical sensors/microphones, manometry, and percussive gastogram. Gut pressure and/or sounds may be measured using, for example, accelerometers and acoustic sensors/microphones.

Sensors 120 may include acoustic, pressure, and/or other types of sensors to identify the presence of high electrical activity but low muscle response indicative of electro-mechanical uncoupling. When electro-mechanical uncoupling occurs, sensors 120, alone or in combination with the other components of monitoring system 100, may measure propagation of slow waves in regions such as the stomach, intestine, and colon.

In certain embodiments, system 100 may be configured to capture data relevant to actual size and depth of tissue, lesions, ulcers, polyps, tumors and/or other abnormalities within the patient. For example, the size of a lesion or ulcer may range from a scale of 100 micrometers to a few centimeters. Software applications 108 may be configured to collect this depth information and to classify the depth as being superficial, submucosal, and/or muscularis. System 100 also be configured to capture data regarding the prevalence of impact of lesions or ulcers within a specific region of the patient.

Data gathered from any of the sources above may be used to train an algorithm, such as an AI algorithm, to predict exacerbations or flare-ups. Information input regarding medication may be used to, for example, predict or otherwise consider a patient's response to medication and enable a health care provider, patient, caregiver or other party to tailor medication treatments. Data from different sources described above may be combined in various permutations in order to enable predictive diagnostics and/or treatment recommendations.

System 100 may further be configured to capture information regarding inflammation. For example, imaging device 104 may be capable of capturing data regarding vasculature including patchy obliteration and/or complete obliteration, dilation or over-perfusion, data related to perfusion information and real-time perfusion information, data relevant to blood's permeation into a tissue or data relevant to tissue thickening, which may be the result of increased blood flow to a tissue and possible obliteration of blood vessels and/or inflammation. Software applications 108 are configured to process this data and compare it to information or data within memory 112 to provide a more accurate diagnosis to the physician.

System 100 may also be configured to measure stenosis in a target lumen within the patient, such as the GI tract, by assessing the amount of narrowing in various regions of the target lumen. System 100 may also be configured to assess, for example, tissue properties such as stiffness. For example, stiffness may be monitored during expansion of a balloon or stent to prevent unwanted fissures or damage.

Imaging device 104 may further be configured to assess bleeding. For example, imaging device 104 may capture data relevant to spots of coagulated blood on a surface of mucosa which can implicate, for example, scarring. Imaging device 104 may also be configured to capture data regarding free liquid in a lumen of the GI tract. Such free liquid may be associated with plasma in blood. Furthermore, imaging device 104 may be configured to capture data relevant to hemorrhagic mucosa and/or obliteration of blood vessels.

Software application 108 may further be configured to process information regarding lesions, ulcers, tumors and/or other tissue abnormalities. For example, software application 108 may also be configured to accurately identify and assess the impact of lesions and/or ulcers on one or more specific regions of the GI tract. For example, software application 108 may compare the relative prevalence of lesions and/or ulcers across different regions of the GI tract. For example, software application 108 may calculate the percentage of affected surface area of a GI tract and compare different regions of the GI tract. As a further example, software application 108 may quantify the number of ulcers and/or lesions in a particular area of the GI tract and compare that number with other areas of the GI tract. Software application 108 may also consider relative severity of ulcers and/or lesions in an area of the GI tract by, for example, classifying one or more ulcers and/or lesions into a particular pre-determined classification, by assigning a point scoring system to ulcers and/or lesions based on severity, or by any other suitable method.

Software application 108, along with one or more imaging devices 104, may be configured to quantify severity of one or more symptoms or characteristics of a disease state. For example, software application 108 may be configured to assign quantitative or otherwise objective measure to one or more disease conditions such as ulcers/lesions, tumors, inflammation, stenosis, and/or bleeding. Software application 108 may also be configured to assign a quantitative or otherwise objective measure to a severity of a disease as a whole. Such quantitative or otherwise objective measures may, for example, be compared to one or more threshold values in order to assess the severity of a disease state. Such quantitative or otherwise objective measures may also be used to take preventative or remedial measures by, for example, administering treatment through a therapy delivery system as discussed below or by providing an alert (e.g., to medical personnel, a patient, or a caregiver).

Software application 108 may store the results or any component of its analyses, such as quantitative or otherwise objective measures, in memory 112. Results or information stored in memory 112 may later be utilized for, for example, tracking disease progression over time. Such results may be used to, for example, predict flare-ups and take preventative or remedial measures by, for example, administering treatment through a therapy delivery system as discussed or by providing an alert (e.g., to medical personnel, a patient, or a caregiver).

Imaging device 104 may be in communication either directly or indirectly with software application 108, which may be stored on a processor or other suitable hardware. Imaging device 104 may be connected with software application 108 by a wired or wireless connection. Alternatively, imaging device 104 may be in communication with another type of processing unit. Software application 108 may run on a specialized device, a general-use smart phone or other portable device, and/or a personal computer. Software application 108 may also be part of an endoscope system, endoscope tool, wireless endoscopic capsule, or implantable device which also includes imaging device 104. Software application 108 may be connected by a wired or wireless connection to imaging device 104, memory 112, therapy delivery system 116 and/or sensors 120.

Imaging device 104 may be configured to capture images at one or more locations at target site(s) within the patient. Imaging device 104, a device carrying imaging device 104, or another component of monitoring system 100, such as software application 108, may be capable of determining the location of the target site where images were recorded. Imaging device 104 may capture images continually or periodically.

Imaging device 104 may be any imaging device capable of taking images including optical, infrared, thermal, or other images. Imaging device 104 may be capable of taking still images, video images, or both still and video images. Imaging device 104 may be configured to transmit images to a receiving device, either through a wired or a wireless connection. Imaging device 104 may be, for example, a component of an endoscope system, a component of a tool deployed in a working port of an endoscope, a wireless endoscopic capsule, or one or more implantable monitors or other devices. In the case of an implantable monitor, such an implantable monitor may be permanently or temporarily implanted.

In certain embodiments, imaging device 104 is an endoscope. The term “endoscope” in the present disclosure refers generally to any scope used on or in a medical application, which includes a body (human or otherwise) and includes, for example, a laparoscope, duodenoscope, endoscopic ultrasound scope, arthroscope, colonoscope, bronchoscopes, enteroscope, cystoscope, laparoscope, laryngoscope, sigmoidoscope, thoracoscope, cardioscope, and saphenous vein harvester with a scope, whether robotic or non-robotic.

When engaged in remote visualization inside the patient's body, a variety of scopes are used. The scope used depends on the degree to which the physician needs to navigate into the body, the type of surgical instruments used in the procedure and the level of invasiveness that is appropriate for the type of procedure. For example, visualization inside the gastrointestinal tract may involve the use of endoscopy in the form of flexible gastroscopes and colonoscopes, endoscopic ultrasound scopes (EUS) and specialty duodenum scopes with lengths that can run many feet and diameters that can exceed 1 centimeter. These scopes can be turned and articulated or steered by the physician as the scope is navigated through the patient. Many of these scopes include one or more working channels for passing and supporting instruments, fluid channels and washing channels for irrigating the tissue and washing the scope, insufflation channels for insufflating to improve navigation and visualization and one or more light guides for illuminating the field of view of the scope.

Smaller and less flexible or rigid scopes, or scopes with a combination of flexibility and rigidity, are also used in medical applications. For example, a smaller, narrower and much shorter scope is used when inspecting a joint and performing arthroscopic surgery, such as surgery on the shoulder or knee. When a surgeon is repairing a meniscal tear in the knee using arthroscopic surgery, a shorter, more rigid scope is usually inserted through a small incision on one side of the knee to visualize the injury, while instruments are passed through incisions on the opposite side of the knee. The instruments can irrigate the scope inside the knee to maintain visualization and to manipulate the tissue to complete the repair

Other scopes may be used for diagnosis and treatment using less invasive endoscopic procedures, including, by way of example, but not limitation, the use of scopes to inspect and treat conditions in the lung (bronchoscopes), mouth (enteroscope), urethra (cystoscope), abdomen and peritoneal cavity (laparoscope), nose and sinus (laryngoscope), anus (sigmoidoscope), chest and thoracic cavity (thoracoscope), and the heart (cardioscope). In addition, robotic medical devices rely on scopes for remote visualization of the areas the robotic device is assessing and treating.

These and other scopes may be inserted through natural orifices (such as the mouth, sinus, ear, urethra, anus and vagina) and through incisions and port-based openings in the patient's skin, cavity, skull, joint, or other medically indicated points of entry. Examples of the diagnostic use of endoscopy with visualization using these medical scopes includes investigating the symptoms of disease, such as maladies of the digestive system (for example, nausea, vomiting, abdominal pain, gastrointestinal bleeding), or confirming a diagnosis, (for example by performing a biopsy for anemia, bleeding, inflammation, and cancer) or surgical treatment of the disease (such as removal of a ruptured appendix or cautery of an endogastric bleed).

As illustrated in FIG. 2, a representative endoscope system 101 has an endoscope 106, a light source device 117, a processor 110, a monitor 111 (display unit), and a console 113. The endoscope 106 is optically connected to the light source device 117 and is electrically connected to the processor device 110. The processor device 110 is electrically connected to the monitor 111 and the console 113. The monitor 111 outputs and displays an image of an observation target, information accompanying the image, and so forth. The console 113 functions as a user interface that receives an input operation of designating a region of interest, setting a function, or the like.

The illumination light emitted by the light source unit 117 passes through a light path coupling unit 119 formed of a mirror, a lens, and the like and then enters a light guide built in the endoscope 106 and a universal cord 115, and causes the illumination light to propagate to the distal end portion 114 of the endoscope 106. The universal cord 115 is a cord that connects the endoscope 106 to the light source device 117 and the processor device 110. A multimode fiber may be used as the light guide.

The hardware structure of a processor 110 executes various processing operations, such as the image processing unit, and may include a central processing unit (CPU), which is a general-purpose processor executing software (program) and functioning as various processing units; a programmable logic device (PLD), which is a processor whose circuit configuration is changeable after manufacturing, such as a field programmable gate array (FPGA); a dedicated electric circuit, which is a processor having a circuit configuration designed exclusively for executing various processing operations, and the like.

FIG. 2 also illustrates a representative endoscope 106 for use with the present disclosure including a proximal handle 127 adapted for manipulation by the surgeon or clinician coupled to an elongate shaft 114 adapted for insertion through a natural orifice or an endoscopic or percutaneous penetration into a body cavity of a patient. Endoscope 100 further includes a fluid delivery system 125 coupled to handle 127 via a universal cord 115. Fluid delivery system 125 may include a number of different tubes coupled to internal lumens within shaft 114 for delivery of fluid(s), such as water and air, suction, and other features that may be desired by the clinician to displace fluid, blood, debris and particulate matter from the field of view. This provides a better view of the underlying tissue or matter for assessment and therapy. In the representative embodiment, fluid delivery system 125 includes a water-jet connector 118, water bottle connector 121, a suction connector 122 and an air pipe 124. Water jet connector 118, water bottle connector 121, suction connector 122 and air pipe 124 are each connected to internal lumens 128, 130, 132, 134 respectively, that pass through shaft 114 to the distal end of endoscope 100.

Endoscope 100 may further include a working channel (not shown) for passing instruments therethrough. The working channel permits passage of instruments down the shaft 114 of endoscope 100 for assessment and treatment of tissue and other matter. Such instruments may include cannula, catheters, stents and stent delivery systems, papillotomes, wires, other imaging devices including mini-scopes, baskets, snares and other devices for use with a scope in a lumen.

Proximal handle 127 may include a variety of controls for the surgeon or clinician to operate fluid delivery system 125. In the representative embodiment, handle 127 include a suction valve 135, and air/water valve 136 and a biopsy valve 138 for extracting tissue samples from the patient. Handle 127 will also include an eyepiece (not shown) coupled to an image capture device (not shown), such as a lens and a light transmitting system. The term “image capture device” as used herein also need not refer to devices that only have lenses or other light directing structure. Instead, for example, the image capture device could be any device that can capture and relay an image, including (i) relay lenses between the objective lens at the distal end of the scope and an eyepiece, (ii) fiber optics, (iii) charge coupled devices (CCD), (iv) complementary metal oxide semiconductor (CMOS) sensors. An image capture device may also be merely a chip for sensing light and generating electrical signals for communication corresponding to the sensed light or other technology for transmitting an image. The image capture device may have a viewing end - where the light is captured. Generally, the image capture device can be any device that can view objects, capture images and/or capture video.

In some embodiments, endoscope 100 includes some form of positioning assembly (e.g., hand controls) attached to a proximal end of the shaft to allow the operator to steer the scope. In other embodiments, the scope is part of a robotic element that provides for steerability and positioning of the scope relative to the desired point to investigate and focus the scope.

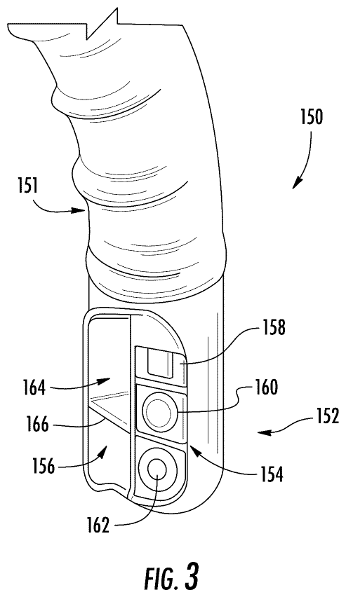

Referring now to FIG. 3, a distal end portion of a side viewing endoscope 150 (e.g., a duodenoscope or EUS) will now be described. As shown, scope 150 includes an elongate flexible shaft 151 with distal end portion 152 having a viewing region 154 and an instrument region 156, both of which face laterally or to the side of the longitudinal axis of shaft 151. Viewing region 154 includes an air nozzle port 158, a camera lens 160 and a light source 162 for providing a view of the surgical site in the patient. Instrument region 156 includes an opening 164 coupled to a working channel (not shown) within shaft 151 of scope 150. Opening 164 is configured to allow passage of instruments from the working channel of scope 150 to the surgical site. Scope 150 also preferably includes an articulation mechanism for adjusting the angle that the instruments pass through opening 164. In the exemplary embodiment, the articulation mechanism comprises an elevator 166, although it will be recognized by those skilled in the art that the articulation mechanism may include a variety of other components designed to articulate the instrument angle, such as a cable extending through shaft 151 or the like.

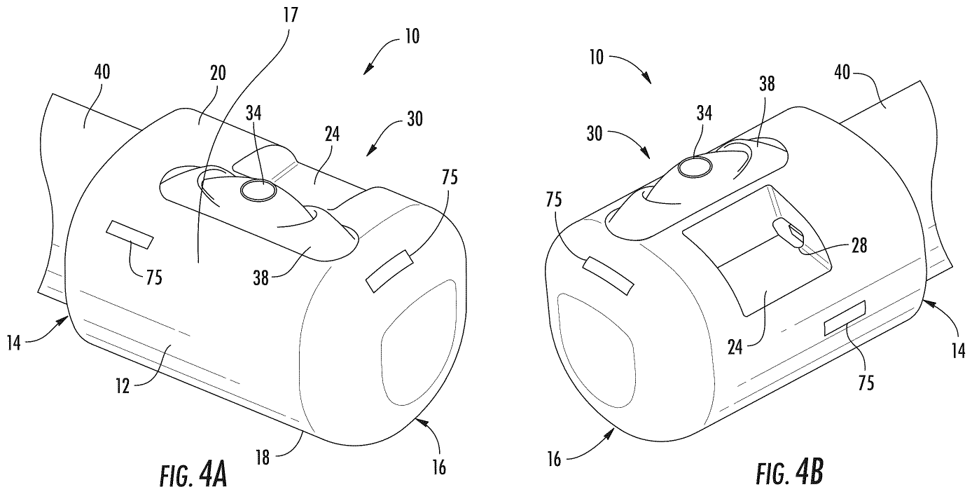

FIGS. 4A and 4B illustrate an exemplary embodiment of a coupler device 10 according to one embodiment of the present disclosure. The coupler device 10 serves as an accessory component for currently existing endoscopes. The device seals and covers infection prone areas of the scope to prevent ingress of debris, fluid, or other unwanted matter that could lead to bacterial contamination and decreased performance of the scope.

As FIGS. 4A and 4B illustrate, the coupler device 10 may comprise a main body 12, proximal end 14 and distal end 16 and an outer surface 17 that includes at least a lower surface 18 and an upper surface 20. The proximal end 14 attaches onto a working end of a duodenum scope 40, extending the working end portion of the scope 40. The upper surface 20 may include a lens and light guide 24 and a scope washer opening 28, which is used to push fluid across the scope camera to wash debris off the camera and is also used to push air across the camera to dry the camera and insufflate the patient's gastrointestinal tract. Upper surface 20 may further include an open area over lens and light guide 24 and scope washer opening 28 to facilitate viewing the surgical site and to allow egress of fluid from scope washer opening 28 into the surgical site (and/or egress of air that may be passed over light guide 24 to dry the camera or that may be passed into the surgical site to insufflate a portion of the site). In addition, the upper surface 20 includes a flexible working channel region 30 that includes a flexible working channel extension 34 that is surrounded by a flexible membrane 38. This flexible membrane 138 serves as a protective hood or covering for the working end of the coupler device 10, providing for flexible articulation while sealing out debris, fluid, bacteria or other unwanted matter.

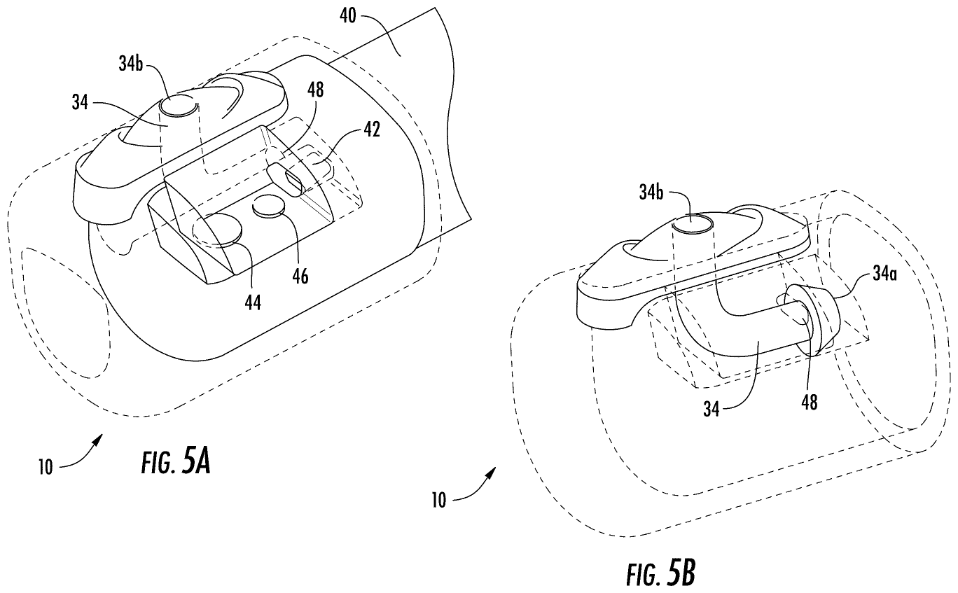

As shown in FIGS. 5A and 5B, the duodenum scope 40 may comprise a light guide 44, lens 46 and washer opening 48. The coupler device 10 cooperates with each of these components of the scope 40 to provide a fully functioning scope. The coupler device 10 does not interfere with the scope's ability to emit a clear image, but instead reduces the risk of contamination with each use. This benefit is achieved by providing a coupler device 10 which attaches to the working end components of the scope 40, and seals around the working end.

According to the present invention, coupler device 10 further includes one or more sensors 75 on, or within, outer surface 17 of main body 12. Sensors 75 are preferably configured to detect one or more physiological parameter(s) of tissue around the outer surface of the main body. As discussed previously, the physiological parameter(s) may include a temperature of the tissue, a type of fluid in, or around, the tissue, pathogens in, or around, the tissue, a dimension of the tissue, a depth of the tissue, a tissue disorder, such as a lesion, tumor, ulcer, polyp or other abnormality, biological receptors in, or around, the tissue, a PH of fluid in, or around the tissue or the like.

In certain embodiments, the coupler device 10 provides a flexible working channel for instruments to be inserted into the scope. The flexible working channel can be angularly adjustable with ease. As shown, in the preferred embodiments, the coupler device 10 may be used with a duodenum scope 40 or other side-viewing scope instrument. It is understood, of course, that the coupler device 10 may be adapted for use with end viewing scopes as well. In addition, the coupler device 10 of the present disclosure can be used with all types of scopes for different medical applications. The duodenum scope 40 shown here is merely for illustrative purposes.

Of course, it will be recognized that the instruments passing through the scope may be articulated by a variety of different mechanism. For example, in some embodiments, the device may have multiple cables so the angle of exit can be articulated in multiple directions, including in different quadrants, unlike with the current endoscope elevators, which can only deflect and therefore redirect an instrument in a single axis due to the limited travel of endoscope elevators, which can only be raised or lowered, but not moved from side to side or articulated into other quadrants. In some embodiments, the cable(s) may be attached directly to the working channel extension or to other devices that can be articulated and cause the working channel extension to change its angle of exit, including, for example, a dowel underneath the working channel extension, but encased in the device that can be advanced forward and backward to move the working channel extension as the cable is advanced and retracted. In some embodiments, the articulation ability of the coupler device may be created with an elevator embedded in the coupler device, which is disposable and therefore thrown away after the procedure.

The articulation ability of the coupler device may also take place with elements that do not involve cables, including for example, piezo electric materials, micro motors, organic semiconductors, and electrically activated polymers. In some embodiments, the articulation ability of the coupler device may also take place with the transfer of force to the working channel extension or an embedded elevator through interlocking connectors that transfer force, wires that twist, slidable sheaths, and memory metals that change shape through the transfer of temperature. In some embodiments, the device includes a power connector or motors to deliver energy, including electromagnetic energy, to the device to cause a transfer in force to change the angle of exit from the coupler device as an instrument is passed through the device, or in advance of passing an instrument through the device. This transfer of force can include causing the device to rotate as it exits the working channel extension. The device may be navigated and articulated by the user directly, or as part of a robotic system in which the users input is translated through the system through various means, including cables, power connectors, motors, electromagnetic energy, slidable sheaths, haptics, computer-guided and directed input, and other means to direct and guide the device to its intended location, including to specific diagnosis and treatment objectives in a patient, or in non-medical applications, to a desired remote location.

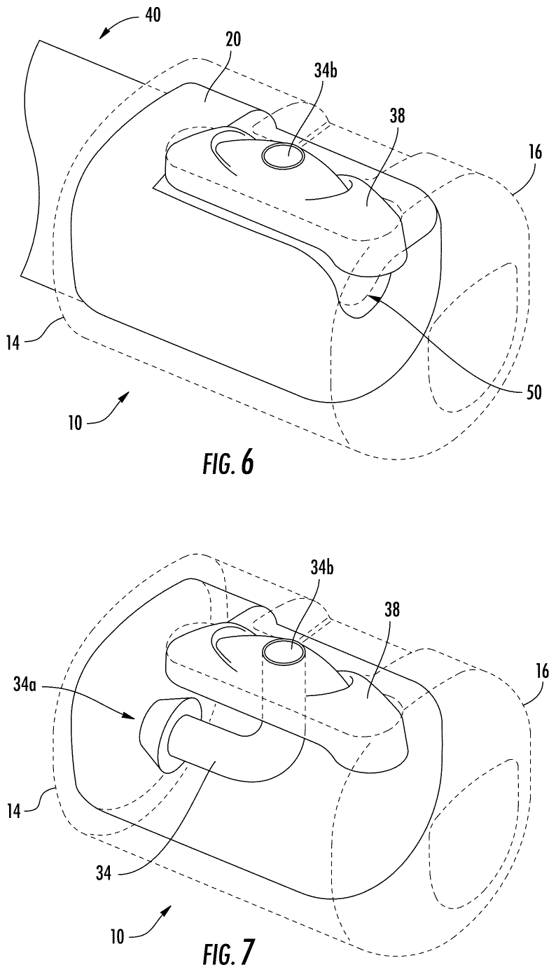

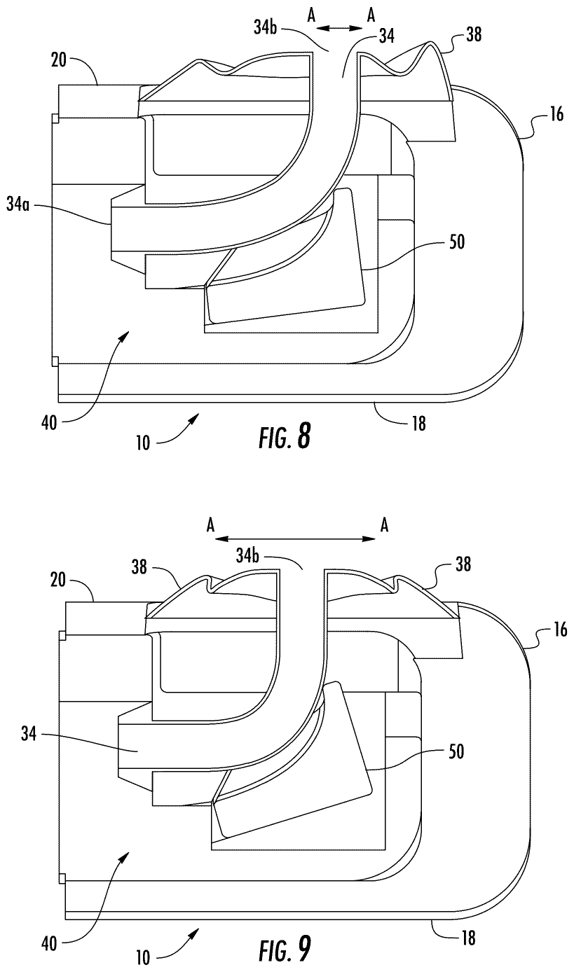

As further shown in FIGS. 4A, 4B, 5A, 5B, 6 and 7, the coupler device 10 provides an extension of the scope's working channel 42. The working channel extension 34 of the coupler device 10 in FIG. 3 is flexible and may contact the scope's working channel 42 by a sealed connection, as shown in FIG. 6, at the proximal end 34a of the working channel extension. The distal end 34b of the working channel extension 34 serves as an exit portal for instruments to pass through the scope 40 to reach different areas of the body.

Additionally, the coupler device 10 provides a further seal around the elevator 50 of the scope. Because the coupler device 10 seals the elevator 40, risk of debris influx, fluids, bacteria and other matter build up behind the elevator and working channel is reduced significantly. This influx of debris, bacteria and other matter is believed to be the reason for drug resistant infections with current scopes today. While preventing influx, the coupler device 10 advantageously maintains flexibility to move the working channel extension 34.

In use, the scope's working channel extension 34 permits passage of instruments down the scope working channel 42 and through and out the working channel extension 34 of the device 40 for assessment and treatment of tissue and other matter. Such instruments may include cannula, catheters, stents and stent delivery systems, papillotomes, wires, other imaging devices including mini-scopes, baskets, snares and other devices for use with a scope in a lumen. This working channel extension 34 is flexible enough that the elevator 50 of the scope 40 can raise and lower the working channel extension 34 so that instruments can be advanced down and out of the working channel extension distal end (or exit portal) 34b of the scope 40 at various angles, or be raised and lowered by a cable or other means to articulate the working channel extension 34.

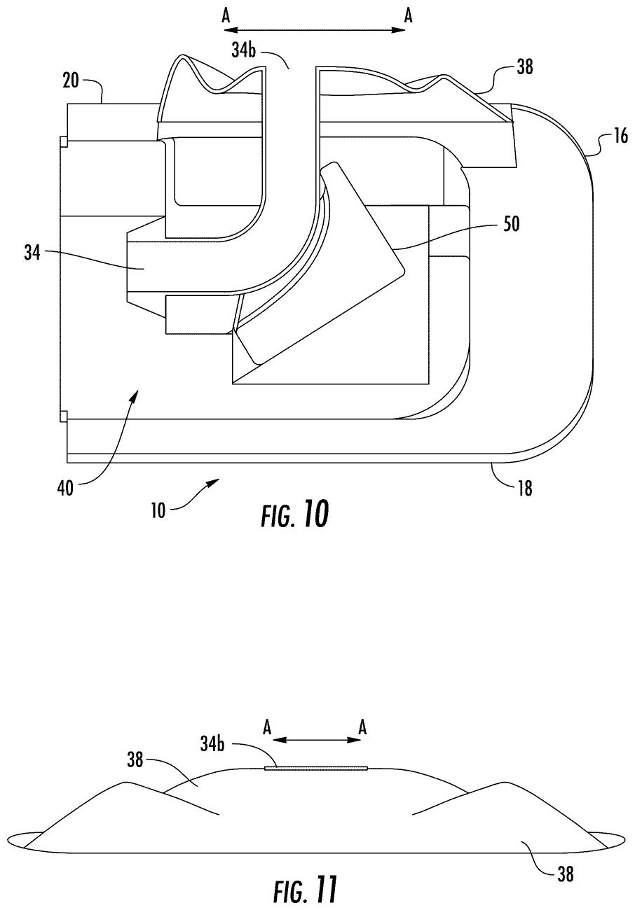

As FIGS. 8 to 10 illustrate, in use when the elevator 50 of the scope 40 is actuated, the flexible working channel extension 34 of the coupler device moves or adjusts to this actuation, along the direction A—A. In FIG. 8, the elevator 50 is raised slightly, creating a hinged ramp or shoulder that pushes the working channel extension 34 a corresponding angle and shifts the exit portal or distal end 34b of the working channel extension to the left. In FIG. 9 the elevator is raised higher than in FIG. 8, such that the distal end 34b of working channel extension 34 is likewise shifted further to the left in comparison to FIG. 8, while FIG. 10 shows the elevator 50 raised even higher and the distal end 34b of working channel extension 34 moved to the left even further in comparison to FIGS. 8 and 9.

As FIG. 11 shows, the ability of the distal end 34b of working channel extension 34 to shift along the width of the working channel region 30 of the coupler device 10 is in part due to the fact that the distal end 34b is itself attached to a flexible membrane 38. This flexible membrane 38 comprises a plurality of loose folds or creases, allowing the excess material to stretch and bend as the elevator actuation forces the working channel extension to bend and shift in response. In addition, the flexible membrane 38 acts as a protective cover or hood for the working channel region 38, preventing the ingress of fluids, debris, or other unwanted matter from getting inside the scope 40 and causing a bacterial contamination or the infusion of other unwanted fluid, debris or particulate matter.

It is contemplated that the coupler device 10 of the present disclosure may be configured for single, disposable use, or it may be configured for reuse. The coupler device 10 may be made of any biocompatible material, such as for example, silicone or another elastic or polymeric material. In addition, the material may be transparent. As shown in FIG. 11, the coupler device 10 may be formed of a transparent material to provide a transparent covering of the scope camera and light source, thereby allowing unhindered performance of the scope 40.

Of course, it will be recognized that the coupler device 10 may be adapted for use with scopes that are actuated by cable and eliminates the need for the elevator component. In these embodiments, the coupler device 10 maintains the same structural features as previously described, but now includes a further disposable external sheath that can receive an interior actuating cable of the scope. This cable can be detached from the elevator and reattached to the flexible working channel extension 34 of the coupler device 10. The elevator is no longer needed in this embodiment, as actuation of the cable effects movement of the working channel extension 34. The external sheath may be configured to attach directly to the scope 40, such as by winding around the outside of the scope or by a friction fit connection. In embodiments, multiple cables may be included in one or more sheaths to provide for articulation in other quadrants than the single axis articulation with elevators in current duodenoscopes. A more complete description of suitable coupler devices for the present disclosure can be found in commonly-assigned, co-pending PCT Patent Application No. PCT/US 2016/043371, filed Jul. 21, 2016, U.S. patent application Ser. No. 16/717,702, filed Dec. 17, 2019 and U.S. patent application Ser. No. 16/717,804, filed Dec. 17, 2019, the complete disclosures of which are incorporated herein by reference in their entirety for all purposes.

In other embodiments, the coupler device 10 may also include a closable port (i.e., self-sealing) that allows for the injection of anti-adhesion, anti-bacterial, anti-inflammatory or other drug or infusible matter that prevents the adherence or colonization of bacteria on the scope. An applicator may be provided that is integrated into the coupler device 10 with a port for delivery of the infusible matter. Alternatively, the applicator may be separate from the coupler device 10 and applied to the distal end of the scope 40. The infusible matter may include forms of silver, including in a gel or other solution, platinum, copper, other anti-adhesion, anti-bacterial, anti-inflammatory or other drug or infusible matter that is compatible with the scope and coupler device materials and biocompatible for patient use.

In one exemplary embodiment, the device includes an anti-infective material. In another exemplary embodiment, the device includes an anti-infective coating. In still another embodiment, the device includes a coating that is hydrophobic. In yet another embodiment, the device is superhydrophobic. In even still another embodiment, the device is anti-infective and hydrophobic. Further yet in another embodiment, the device is anti-infective and superhydrophobic. In further still another exemplary embodiment, anti-inflammatory coatings are incorporated into the device. In other embodiments, the anti-inflammatory coating may be hydrophilic.

In one exemplary embodiment, the device 10 may include a silver ion coating. In another embodiment, the device 10 may have a silver hydrogel applied, infused, or made part of the device 10 in the area that covers or goes around the scope elevators. In addition to silver having antimicrobial properties, silver can also conduct electricity. Thus, in still another embodiment, the device 10 may include an electrical wire or other power transmission point to enable the creation of an electric field across the silver ion coating to improve the ability of the silver ion coating to prevent infection. In some embodiments, the electrical wire or other power transmission point may also apply to other antimicrobial and conductive materials, including platinum and copper.

The working channel extensions described herein may comprise a combination of different materials. For example, the working channel extension may be formed of multiple elastic materials joined to a biocompatible metal. In some embodiments, one of the elastic materials may be PTFE and another elastic material may be a biocompatible elastic material that covers the biocompatible metal. The working channel extension may comprise an inner elastic material and an outer elastic material. The outside of the working channel extension may include a biocompatible metal 230, which may take the form of a coil or winding. In one embodiment, the biocompatible metal may be encapsulated by one or more of the elastic materials.

The outer biocompatible elastic material may be formed to create a gasket to seal the proximal end of the working channel extension against the working channel of an endoscope, creating a seal to prevent the intrusion of unwanted bacteria, biomatter and other material into this sealed area.

The working channel extension may include an adjustable angle of exit for locking an instrument in place. In this embodiment, when the angle of exit is adjusted, it creates compressive force in the working channel, locking an instrument in place. This can be used to fixate an instrument while a wire is advanced through the instrument, or to fixate a wire, while a second instrument is exchanged over the wire.

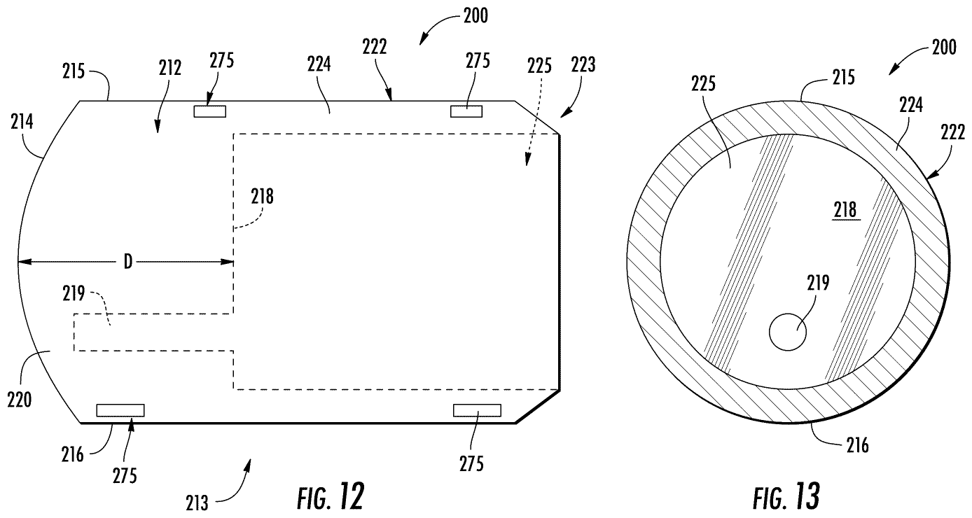

Turning now to FIGS. 12-14, there is shown an alternative embodiment of an optical coupler 200 according to the invention. The optical coupler 200 includes a visualization section 212 at a distal end 213 of the optical coupler 200. The visualization section 212 has a generally slightly curved, convex outer surface 214 that extends from a first outer side boundary 215 to a second opposite outer side boundary 216 of the optical coupler 200. The outer surface 214 may be constructed to be generally flat, but a curved outer surface 214 is preferable because the curvature helps to clear the field of view by pushing any fluid or matter from the center of the outer surface 214 to the outer boundaries 215, 216. A flat outer surface 214 may be more difficult to clear since the pressure is equal across the entire area of contact and fluid can become trapped between the lens and a surface in which it is desired to view or perform work. A curved outer surface 214 is also preferable to correct any curvature distortion created by an objective lens that may be used in conjunction with the coupler 200.

The optical coupler 200 has a proximal surface 218, and a hollow instrument channel 219 extends from the proximal surface 218 toward the outer surface 214. The hollow instrument channel 219 may be constructed such that the channel 219 does not extend all the way through the visualization section 212 to the outer surface 214. In such a case, a barrier section 220 of material is provided between a distal end 221 of the hollow instrument channel 219 and the outer surface 214 of the optical coupler 200. Alternatively, the instrument channel 219 may extend the full length of the visualization section 212, extending through the optical coupler 200. Such a configuration may allow for the free and unencumbered exchange of instruments. A water tight seal or valve, such as a Tuohy-Borsttype valve, may be employed on the proximal end of the endoscope instrument channel 219 to prevent or minimize air, fluid, and/or foreign matter from flowing through the instrument channel 19.

While an instrument channel 219 is shown in the optical coupler 200 of FIGS. 12-14, the visualization section 212 may be constructed without an instrument channel 219. In such a case, instruments may be passed directly through the visualization section 212 as the visualization section 212 may be constructed of a material that is self-sealing and elastic enough to permit instruments to be passed through the entire length of the visualization section 212 of the optical coupler 200. An example of an optical coupler 200 without an instrument channel 219 is described in U.S. Pat. No. 8,905,921 to Titus, the complete disclosure of which is hereby incorporated herein by reference in its entirety for all purposes.

The optical coupler 200 also includes an attachment section 222 connected to and extending away from the visualization section 212. The attachment section 222 is at the proximal end 223 of the optical coupler 200. The proximal end 223 of the optical coupler may be angled to lessen the chance that the optical coupler 200 may catch on any surfaces when the optical coupler 200 is being removed from its environment of use. In the embodiment shown, the attachment section 222 is in the form of a cylindrical wall 224. The proximal surface 218 and the cylindrical wall 224 of the optical coupler 200 define a hollow cylindrical opening 225 of the optical coupler 200 within the sleeve-like cylindrical wall 224.

Optical coupler 200 further includes one or more sensors 275 on, or within, visualization section 212 and/or attachment section 222. Sensors 75 are preferably configured to detect one or more physiological parameter(s) of tissue around the outer surface of the main body. As discussed previously, the physiological parameter(s) may include a temperature of the tissue, a type of fluid in, or around, the tissue, pathogens in, or around, the tissue, a dimension of the tissue, a depth of the tissue, a tissue disorder, such as a lesion, tumor, ulcer, polyp or other abnormality, biological receptors in, or around, the tissue, a PH of fluid in, or around the tissue or the like.

Optical coupler 200 may further include an ultrasound transducer or sensor (not shown) mounted in either attachment section 222 or visualization section 212. The ultrasound transducer may be a transmitter, receiver or a transceiver. The electrical signal may be transmitted to the ultrasound transducer through a connector that extends through the endoscope, or wirelessly through the patient's body. The transducer measures the time between sending a sound signal and receiving the echo of the sound signal to calculate the distance therebetween. This data can be collected by a processor coupled to the endoscope, or wirelessly directly to the optical coupler 200, to measure depth of tissue and create a 3-D representation of a target area within the patient.

Optical coupler 200, or the endoscope, may also include a laser or other light transmitter (not shown) for transmitting ultraviolet, visible and/or infrared light against target tissue. In this embodiment, sensors 275 are configured to detect the reflected light (i.e., light return times and/or wavelengths) and to transmit signals related to the reflected light to the processor. This data can be used to create a 3-D representation of the target tissue.

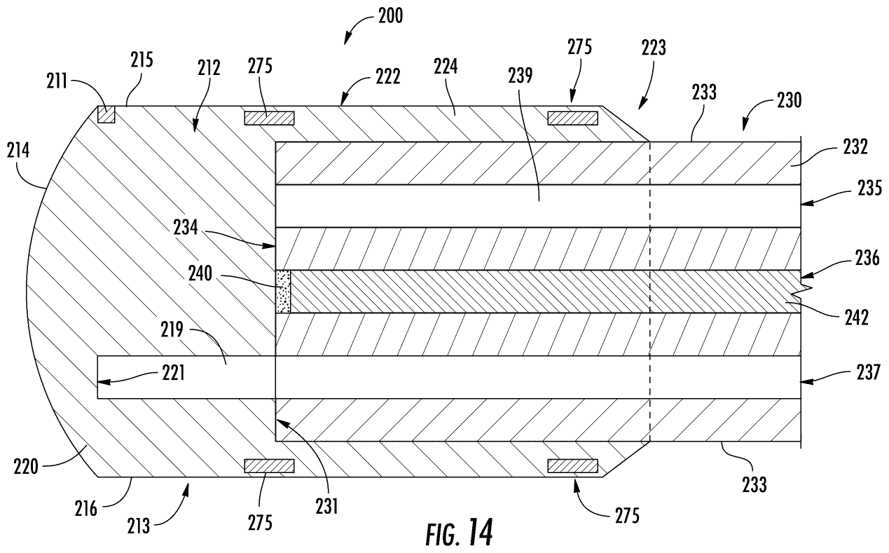

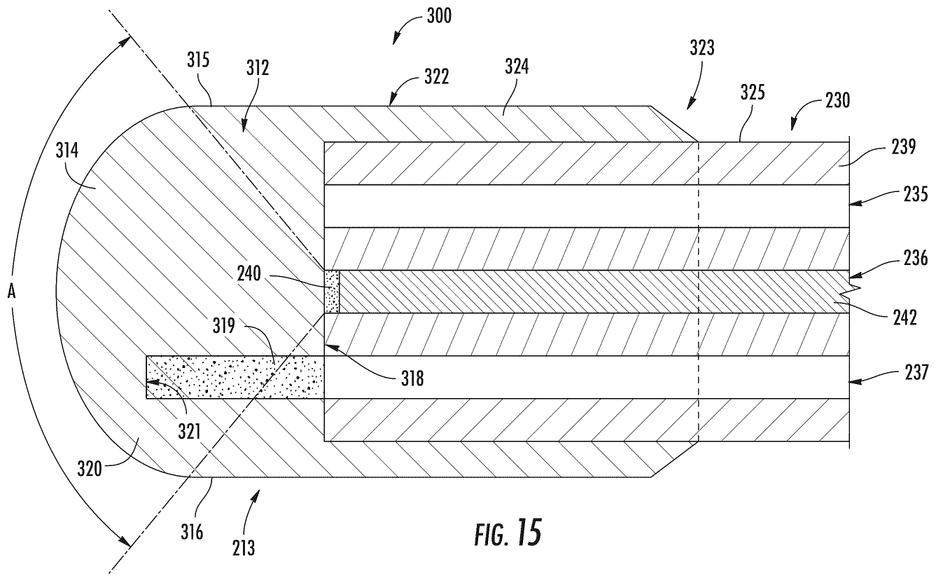

Referring to FIG. 14, the optical coupler 200 can be mounted on an endoscope 230. The endoscope 230 has a distal end 231 that is inserted in the hollow cylindrical opening 225 of the optical coupler 200. In one form, the cylindrical wall 224 of the coupler 200 has a diameter one to three millimeters larger than the endoscope 230. The endoscope 230 has a sheath 232 with an outer surface 233 that snugly engages the cylindrical wall 224 of the optical coupler 200. In a non-limiting example, the sheath 232 has an outside diameter of 7-15 millimeters. An end surface 234 of the endoscope 230 sealingly engages the proximal surface 218 of the optical coupler 200.

The endoscope 230 includes a first lumen 235 and a second lumen 236 and a third lumen 237 that extend from the end surface 234 of the endoscope 230 to a proximal end (not shown) of the endoscope. Lumen internal diameters of 2-4 millimeters are typical. A light guide 239 is positioned in the first lumen 235 for transmitting light toward a surface area at or beyond the outer surface 214 of the optical coupler 200.

An object lens 240 is positioned at a distal end of an image carrying fiber 242, and the lens 240 is optically connected to the image carrying fiber 42 for receiving light that has been reflected from the surface area being viewed. The object lens 240 and the image carrying fiber 242 are located in the second lumen 236. The third lumen 237 aligns with the hollow instrument channel 219 of the optical coupler 200 when the optical coupler 200 is mounted on the endoscope 230. In the embodiment shown, the instrument channel 219 and the third lumen 237 have the same size inner diameter within a tolerance of ±5%. The optical coupler 20 can also include a Light Emitting Diode (LED) 211 near the outer surface 214 of the coupler to provide illumination prior to the coupler contacting any fluids, tissue, or structure. The LED 211 may be provided power via a wire (not shown) in the endoscope 230 or from an external source.

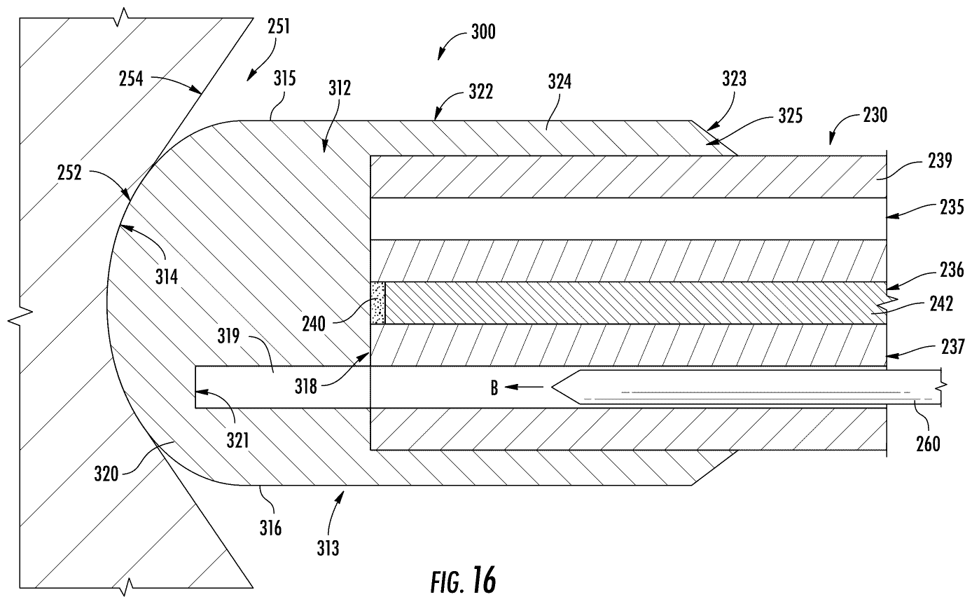

In one example configuration, the endoscope 230 may be a fixed-focus endoscope having a specific depth of field. The outer surface 214 may be spaced apart from the proximal surface 218 of the optical coupler 200 by a length D (see FIG. 15) equal to a reference distance selected from values in the depth of field distance range of the endoscope 200. In one example configuration, the endoscope 200 may have a depth of field in the range of 2 to 100 millimeters. In this case, the outer surface 214 is spaced apart from the proximal surface 218 of the optical coupler 200 by a length in the range 2 to 100 millimeters. Preferably, the length D equals a reference distance that is in the lower 25% of values in the depth of field distance range of the endoscope 200. In one example configuration, the endoscope 200 may have a depth of field in the range of 2 to 100 millimeters. In this case, the length D equals a value of 2-26 millimeters. More preferably, the length D equals a reference distance that is in the lower 10% of values in the depth of field distance range of the endoscope 230.

In one example configuration, the endoscope 230 may have a depth of field in the range of 2 to 100 millimeters. In this case, the length D equals a value of 2-13 millimeters. Most preferably, the length D equals a reference distance that is greater than or equal to the lowest value (e.g., 2 millimeters) in the depth of field distance range of the endoscope 230. In one version of the coupler 10, the length D is 7-10 millimeters, or a typical distance that the endoscope 30 is held from tissue that would be receiving an endoscopic treatment or therapy.

The design of the length D for the optical coupler 200 should also take into consideration the characteristics of the materials that compose the coupler 200, such as any possible compression of the coupler 200 when it is held against a surface. For example, if the coupler 200 may be compressed 1 millimeter when held against a surface and the lowest value in the depth of field distance range of the endoscope 230 is 2 millimeters, then the length D should be greater than or equal to 3 millimeters to compensate for this possible compression.

The optical coupler 200 can be formed from a variety of materials. In one version of the optical coupler 200, the optical coupler 200 is molded from a material selected from silicone gels, silicone elastomers, epoxies, polyurethanes, and mixtures thereof. The silicone gels can be lightly cross-linked polysiloxane (e.g., polydimethylsiloxane) fluids, where the cross-link is introduced through a multifunctional silane. The silicone elastomers can be cross-linked fluids whose three-dimensional structure is much more intricate than a gel as there is very little free fluid in the matrix. In another version of the optical coupler 200, the material is selected from hydrogels such as polyvinyl alcohol, poly(hydroxyethyl methacrylate), polyethylene glycol, poly(methacrylic acid), and mixtures thereof. The material for the optical coupler 200 may also be selected from albumin based gels, mineral oil based gels, polyisoprene, or polybutadiene. Preferably, the material is viscoelastic.

Referring back to FIGS. 12-14, in the optical coupler 200, the material is optically clear such that the light guide 239 can transmit light through the optical coupler 200 toward a surface area at or beyond the outer surface 214 of the optical coupler 200 and such that the optical coupler 200 is capable of transmitting an optical image of the surface area being viewed back to the lens 240. In one version of the optical coupler 200, the material has a degree of light transmittance greater than 80% based on test standard ASTM D-1003 (Standard Test Method for Haze and Luminous Transmittance of Transparent Plastics). In another version of the optical coupler 200, the material has a degree of light transmittance greater than 90% based on test standard ASTM D-1003. In another version of the optical coupler 200, the material has a degree of light transmittance greater than 95% based on test standard ASTM D-1003. In another version of the optical coupler 200, the material has a degree of light transmittance greater than 98% based on test standard ASTM D-1003. Preferably, the material has an optical absorption of less than 0.1% in the visible light range, and more preferably the material has an optical absorption of less than 0.01% in the visible light range. The material has an index of refraction of about 1.3 to about 1.7, and preferably, the index of refraction of the material matches the index of refraction of the light guide 39, or is as low as possible.

The optical coupler 200 may also be coated with different materials to reduce the amount of adherence properties. Additionally, some coatings of the optical coupler 200 improve with light reflections. Sample coatings that may be used on the optical coupler include thermoplastic film polymer based on p-xylylene such as Parylene C, which is an optically clear biocompatible polymer having abrasion resistant and hydrophobic properties.

The hardness of the material of the optical coupler 200 can be varied depending on the application. If the surface being viewed has steep undulations, a very low durometer (soft) surface of the coupler will form to the shape of the object. Alternatively, the coupler could comprise a high durometer (stiff) material to allow the tissue to conform to the shape of the coupler. In one form, the material has a durometer ranging from 2-95 on the Shore 00 scale.

In another form, the material has a durometer ranging from 2-20 on the Shore 00 scale. In another form, the material has a durometer ranging from 40-80 on the Shore 00 scale. In another form, the material has a durometer ranging from 60-80 on the Shore 00 scale. As alluded to above, the material in some applications may preferably have a durometer outside of the ranges of the Shore 00 scale just discussed. Although materials having a hardness of 80 or more on the Shore 00 scale may not technically be considered a “gel”, this specification generally refers to the materials that can compose the coupler 200 by using the term “gel.” The use of the term “gel” is not meant to limit the invention to specific materials or specific ranges of hardness on the Shore 00 scale.

Referring now to FIGS. 15-17, a second embodiment of an optical coupler 300 is shown. In this embodiment, the visualization section 312 may have an outer surface 314 with a greater degree of curvature than the embodiment shown in FIGS. 12-14. The convex, generally dome shaped outer surface 314 extends from a first outer side boundary 315 to a second opposite outer side boundary 316 of the optical coupler 300. The optical coupler 300 has a proximal surface 318, and a hollow instrument channel 319 extends from the proximal surface 318 toward the outer surface 314. A barrier section 320 of material is provided between a distal end 321 of the hollow instrument channel 319 and the outer surface 314 of the optical coupler 300. Preferably, all of the visualization section 312 (other than the hollow instrument channel 319) is a non-porous solid viscoelastic material.

The optical coupler 300 also includes an attachment section 322 connected to and extending away from the visualization section 312. The attachment section 322 is at the proximal end 323 of the optical coupler 300. In the embodiment shown, the attachment section 322 is in the form of a cylindrical wall 324. The proximal surface 318 and the cylindrical wall 324 of the optical coupler 300 define a hollow cylindrical opening 325 of the optical coupler 300.

The optical coupler 300 can be mounted on an endoscope 230. The endoscope 230 has a distal end 231 that is inserted in the hollow cylindrical opening 325 of the optical coupler 300. The endoscope 230 has a sheath 232 with an outer surface 233 that snugly engages the cylindrical wall 324 of the optical coupler 300. An end surface 234 of the endoscope 230 sealingly engages the proximal surface 318 of the optical coupler 300.

The endoscope 230 includes a first lumen 235 and a second lumen 236 and a third lumen 237 that extend from the end surface 234 of the endoscope 230 to a proximal end (not shown) of the endoscope. A light guide 239 is positioned in the first lumen 235 for transmitting light toward a surface area at or beyond the outer surface 314 of the optical coupler 300. An object lens 240 is positioned at a distal end of an image carrying fiber 242, and the lens 240 is optically connected to the image carrying fiber 242 for receiving light that has been reflected from the surface area.

The object lens 240 and the image carrying fiber 242 are located in the second lumen 236. The third lumen 237 aligns with the hollow instrument channel 319 of the optical coupler 300 when the optical coupler 300 is mounted on the endoscope 230. In the embodiment shown, the instrument channel 219 and the third lumen 237 have the same size inner diameter within a tolerance of ±5%.

The endoscope 230 can have a field of view of A degrees (e.g., 90-170°) as shown in FIG. 15. In FIG. 15, a portion of the outer surface 314 of the visualization section 312 is dome-shaped, and the portion of the outer surface 314 of the visualization section 312 that is dome-shaped is within the field of view of the endoscope 230. This provides for improved imaging with an increased working space as organs can be pushed out of the field of view.

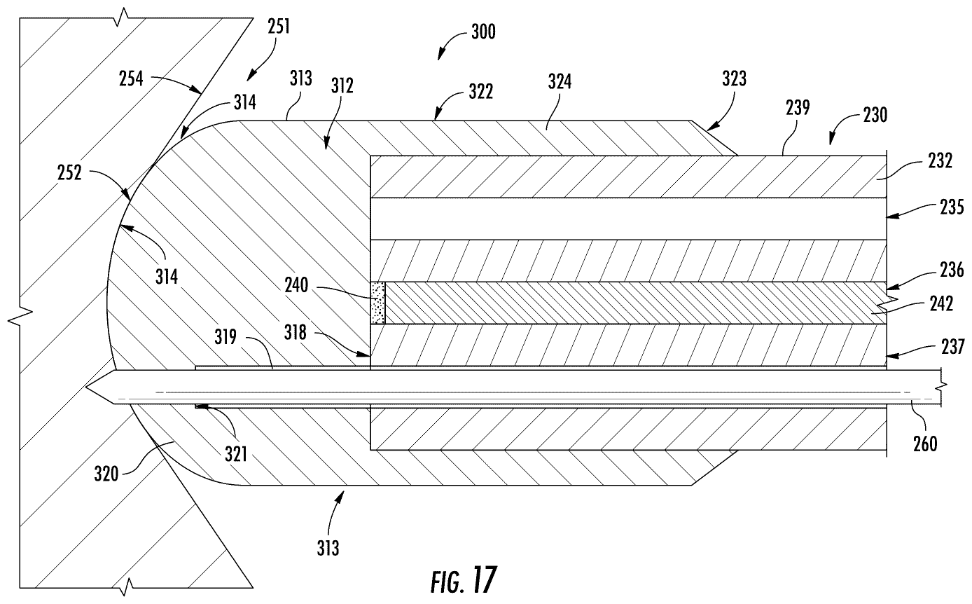

Still referring to FIGS. 16 and 17, after the physician mounts the optical coupler 300 on the endoscope 230, the endoscope is inserted into a body cavity 251. The optical coupler 300 is placed in contact with a region 252 of the wall 254 of the body cavity 251 thereby displacing opaque fluid and/or particulate matter in contact with or adjacent the region. Light is transmitted from a light source through the light guide 239 in a conventional manner. The light then passes through the optical coupler 300 and onto the region 252. Reflected light then passes back through the optical coupler 300 and the lens 240 receives the reflected light from the region 252. The lens 240 transmits an optical image to the image carrying fiber 242 which transmits the optical image to an eyepiece or video display in a conventional manner.

The physician then inserts a medical instrument 260 in direction B (see FIG. 16) in the third lumen 237 of the sheath 232 of the endoscope 230. The medical instrument 260 is passed through the instrument channel 319 in the coupler 300 and then the medical instrument 260 is pierced through the barrier section 320 and the outer surface 314 of the coupler 300. A medical procedure can then be performed using the medical instrument 260 on the region 252 of the wall 254 of the body cavity 251. Non-limiting examples of the medical instrument 260 include a biopsy forceps, an electrocauterization device, an ablation device, and a suturing or stapling device. Optionally, viewing optics can be pierced through the barrier section 320 and the outer surface 314 of the coupler 300.

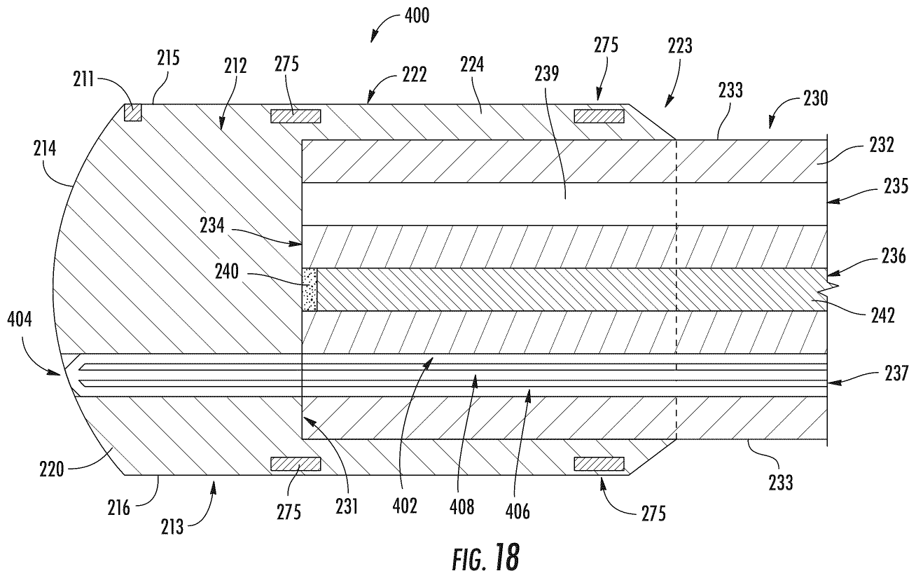

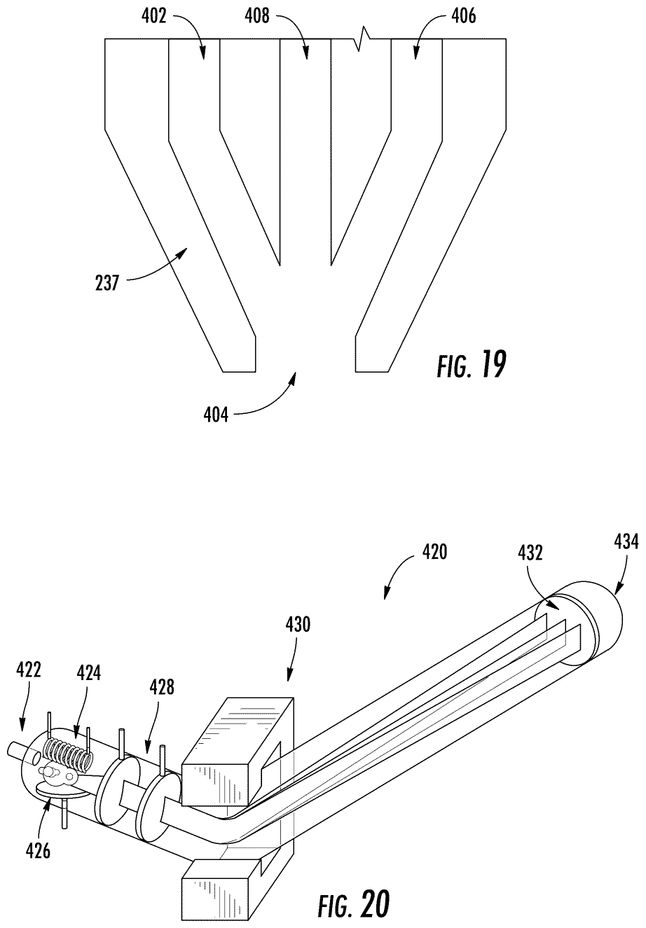

Referring now to FIGS. 18 and 19, a third embodiment of an optical coupler 400 is shown. Optical coupler 400 has many of the same features as the optical couplers in the first two embodiments discussed above. In this embodiment, instrument lumen 237 (or a separate lumen not shown) is a fluid sample lumen configured to withdraw tissue and/or fluid samples from the patient for analysis. To that end, fluid sample lumen 237 comprises a first interior passage or lumen 402 extending through scope 230 and optical coupler 400 to a distal opening 404 at distal surface 214 of the coupler 400. First passage 402 has a proximal end coupled to a fluid delivery system (not shown) for delivering a fluid, such as water, through distal opening 404 to a target site on the patient's tissue. In certain embodiments, fluid delivery system is configured to delivery one or more droplets of water through distal opening 404.

Fluid sample lumen 237 further includes a second interior passage or lumen 406 coupled to distal opening 404. Second passage 406 has a proximal end coupled to an aspiration system (not shown) for withdrawing the water droplets delivered through first lumen 402 along with other fluid and/or tissue from the target site. A third central passage 408 is also provided between first and second passages 402, 406 that is also coupled to distal opening 404. In certain embodiments, central passage 408 has a proximal end coupled to a gas delivery system configured to deliver a gas through central passage 408 to distal opening 404 such that the gas interacts with the fluid droplets and the tissue or fluid sample from the patient.

In a preferred embodiment, the fluid droplets and the gas are delivered to distal opening 408 so as to collect small molecules from the tissue or fluid sample of the patient. These small molecules are then withdrawn from the patient through second passage 406. Of course, it will be recognized that the present invention is not limited to the configuration of fluid sample lumen 237 shown in FIG. 18. For example, fluid sample lumen 237 may extend to one of the peripheral surfaces of optical coupler 200 rather than distal surface 214. In other embodiments of the invention, the fluid sample lumen 237 extends only to the distal end 234 of scope 230. In these embodiments, optical coupler 200 is not attached to scope 230 during the tissue removal process. In still other embodiments, fluid sample lumen 237 may be incorporated into the coupler device shown in FIGS. 4-11.

The fluid or tissue sample withdrawn through fluid sample lumen 237 may be analyzed by a variety of different tissue analyzing devices 118 known in the art, such as a mass spectrometer, cold vapor atomic absorption or fluorescence devices, histopathologic devices and the like. In a preferred embodiment, tissue analyzing device 118 includes a particle detector, such as mass analyzer or mass spectrometer, coupled to the ionizer and configured to sort the ions, preferably based on a mass-to-charge ratio, and a detector coupled to the mass analyzer and configured to measure a quantity of each of the ions after they have been sorted. Monitoring system 100 further comprises one or more software application(s) coupled to the detector and configured to characterize a medical condition of the patient based on the quantity of each of the ions in the tissue sample. The medical condition may include a variety of disorders, such as tumors, polyps, ulcers, diseased tissue, pathogens or the like. In one embodiment, the medical condition comprises a tumor and the processor is configured to diagnose the tumor based on the quantity of each of the ions retrieved from the tissue sample. For example, the processor may be configured to determine the type of proteins or peptides existing in a tissue sample based on the type and quantity of ions. Certain proteins or peptides may provide information to the processor that the tissue sample is, for example, cancerous or pre-cancerous.

Referring now to FIG. 20, a representative particle detector 420, such as a mass spectrometer, may be coupled to fluid sample lumen 237 to analyze the tissue or fluid sample withdrawn from the patient. Particle detector 420 includes a proximal port 422 coupled to second passage 406 of lumen 237 for delivering the tissue or fluid sample into the detector 420. Detector 420 further comprises a heating device 424 configured to vaporize the tissue sample and an ionization source, such as an electron beam 426 or other suitable ionizing device to ionize the vaporized tissue sample by giving the molecules in the tissue sample a positive electric charge (i.e., either by removing an electron or adding a proton). Alternatively, the heating device 424 and/or electron beam 426 may be incorporated directly into optical coupler 200 or scope 230 so that the tissue sample is vaporized and/or ionized before it is withdrawn from scope 230.

Particle detector 420 further includes a mass analyzer for separating the ionized fragments of the tissue sample according to their masses. In one embodiment, the mass analyzer comprises a particle accelerator 428 and a magnet 430 configured to create a magnetic field sufficient to separate the accelerated particles based on their mass/charge ratios. Particle detector 420 further comprises a detector 432 at a distal end 434 of particle detector for detecting and transmitting data regarding the various particles from the tissue sample.

According to the present disclosure, a software application 108, such as the machine-learning or artificial intelligent software application described above, may be coupled to particle detector 420 to analyze the detected particles. For example, the software application may determine the type of proteins or peptides within the tissue sample based on their mass-to-charge ratios. The software application may further determine, based on data within memory 112, whether the proteins or peptides indicate cancerous tissue in the patient. Alternatively, software application 108 may determine molecular lesions such as genetic mutations and epigenetic changes that can lead cells to progress into a cytologically preneoplastic or premalignant form.

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

What is claimed is:

1. A system for recognizing a medical condition in a patient, the system comprising:
an imaging device having a light source and a camera for capturing images of a tissue in the patient, the imaging device comprising an internal working channel;
a coupler device removably coupled to a distal end of the imaging device, the coupler device comprising one or more sensors for detecting a physiological parameter of tissue around the coupler device, wherein the physiological parameter comprises one of a temperature, a type of fluid around the tissue, a presence of pathogens, a dimension of the tissue, a presence of a biological receptor, and a PH of fluid around the tissue, wherein the coupler device comprises an internal passage having an open distal end and a proximal end configured to be aligned with the internal working channel of the imaging device when the coupler device is coupled to the distal end of the imaging device; and
a processor coupled to the imaging device and having a software application with a first set of instructions for recognizing the images captured by the imaging device and a second set of instructions for determining if the tissue contains a medical condition based on the images and the physiological parameter detected by the one or more sensors.

2. The system of claim 1, further comprising a memory in communication with the processor, wherein the memory contains images of representative tissue and wherein the second set of instructions causes the processor to compare the images of the tissue captured by the imaging device with the images of representative tissue.

3. The system of claim 2, wherein the software application is configured to develop from the images of representative tissue at least one set of computer-executable rules useable to recognize a medical condition in the tissue images captured by the optical imaging device.

4. The system of claim 3, further comprising an artificial neural network coupled to the processor comprising at least one trained machine learning algorithm configured to recognize the medical condition based on the images of representative tissue.

5. The system of claim 2, wherein the images of representative tissue include images from patients with known medical conditions, disorders or diseases.

6. The system of claim 2 wherein the memory contains images of tissue from previous surgeries on the patient.

7. The system of claim 1, wherein the second set of instructions causes the processor to identify objects in the tissue images and wherein the memory includes a set of representative objects associated with the medical condition.

8. The system of claim 7, wherein the software application includes a third set of instructions for comparing the objects in the tissue images with the representative objects to determine if the tissue contains a medical condition.

9. The system of claim 2, wherein the second set of instructions causes the processor recognize abnormalities in the tissue based on characteristics of the representative tissue.

10. The system of claim 9, wherein the abnormalities are selected from the group consisting essentially of tissue color, tissue texture, tissue shape, tissue size and tissue topography.

11. The system of claim 1, wherein the second set of instructions causes the processor to determine if the tissue deviates from a threshold value.

12. The system of claim 1, wherein the processor includes a third set of instructions for determining a differentiation value of the tissue images, wherein the differentiation value provides a quantitative measure for a grade or level of development of the medical condition, disorder or disease.

13. The system of claim 1, wherein the medical condition is a cancerous tissue, a tumor, a polyp, an ulcer, a lesion, an inflammation or a diseased tissue.

14. The system of claim 2, wherein the images of representative tissue comprises a topographic representation of tissue within a target area of the patient.

15. The system of claim 1, wherein the imaging device is an optical imaging device.

16. The system of claim 15, wherein the coupler device comprises a main body having a visualization section configured to allow viewing of the surgical site, and an attachment section having a proximal end configured for removable attachment to a distal end portion of the optical imaging device.

17. The system of claim 1, wherein the memory contains data regarding physiological parameters of known medical conditions and wherein the software application comprises a third set of instructions for recognizing the medical condition in the patient based on the physiological parameter detected by the sensor and the physiological parameters of known medical conditions.

18. The system of claim 17, further comprising an artificial neural network coupled to the processor comprising at least one trained machine learning algorithm configured to develop from the physiological parameters at least one set of computer-executable rules useable to recognize a medical condition in the physiological parameters detected by the one or more sensors sensor.

19. A system for using machine learning to recognize a medical condition in a patient, the system comprising:
an endoscope configure to collect optical images of the patient and comprising an internal working channel;
a coupler device removably coupled to a distal end of the endoscope, the coupler device comprising one or more sensors for detecting a physiological parameter of tissue around the coupler device, wherein the coupler device comprises an internal passage having an open distal end and a proximal end configured to be aligned with the internal working channel of the imaging device when the coupler device is coupled to the distal end of the imagine device;
a processor with a software application having at least one trained machine learning algorithm;
a memory in communication with the processor and containing images of representative tissue;
wherein the software application has a first set of instructions for recognizing the optical images of tissue in the patient and a second set of instructions for causing the processor to compare the optical images of the tissue in the patient with the images of representative tissue; and
wherein the trained machine learning algorithm is configured to develop from the images of representative tissue and the physiological parameter at least one set of computer-executable rules useable to recognize the medical condition in the optical images of the tissue in the patient.
